(19) United States
(12) Patent Application Publication
(10) Pub. No.: US 2026/0060977 A1
(43) Pub. Date: Mar. 5, 2026

(54) COMPOSITIONS AND METHODS FOR TREATING PROTOPORPHYRIA

(71) Applicant: RUTGERS, THE STATE UNIVERSITY OF NEW JERSEY, New Brunswick, NJ (US)

(72) Inventors: Bishr Omary, New Brunswick, NJ (US); Ning Kuo, New Brunswick, NJ (US); Raheleh Nasrollahi, New Brunswick, NJ (US)

(21) Appl. No.: 19/311,696

(22) Filed: Aug. 27, 2025

Related U.S. Application Data
(60) Provisional application No. 63/688,019, filed on Aug. 28, 2024.

(51) Int. Cl. A61K 31/495; A61K 31/341; A61K 31/4164; A61K 31/445; A61P 1/16

(52) U.S. Cl. CPC A61K 31/495; A61K 31/341; A61K 31/4164; A61K 31/445; A61P 1/16

(57) ABSTRACT

Certain embodiments of the invention provide a method of lowering Protoporphyrin IX (PP-IX) level or treating protoporphyria (e.g., EPP) in a subject in need of, comprising administering one or more antihistamines (e.g., administering an H1 antihistamine, or administering two antihistamines such as an H1 antihistamine and an H2 antihistamine) to the subject.

### CROSS-REFERENCE TO RELATED APPLICATION

This application claims priority to U.S. Provisional Application No. 63/688,019 that was filed on Aug. 28, 2024. The entire content of the application referenced above is hereby incorporated by reference herein.

### STATEMENT OF GOVERNMENT SUPPORT

This invention was made with government support under DK116548 awarded by the National Institutes of Health. The government has certain rights in the invention.

### BACKGROUND

Erythropoietic protoporphyria (EPP) is a skin photosensitivity and other potential internal organ disorder caused by the loss of ferrochelatase activity below 30% of normal, leading to accumulation of protoporphyrin-IX (PP-IX) in the liver and in bone marrow erythroid precursors. Skin photosensitivity is a major manifestation of EPP with 10-25% of EPP patients developing liver dysfunction and gallstones and 1-4% of overall cases progressing to end-stage liver failure needing liver transplantation, and gallstones in up to 30% of patients. X-linked protoporphyria (XLP) also leads to PP-IX accumulation due to a gain of function mutation in the enzyme 5′-aminolevulinate synthase 2 (ALAS2 or erythroid ALA-synthase) that is found in developing erythrocytes. Patients with XLP also develop photosensitivity and hepatobiliary disease. Despite drugs that may relieve some of the skin symptoms to some extent for certain patients, reliable therapeutics for EPP/XLP-related liver disease remains a major unmet need. Therefore, effective treatments for EPP, XLP, and EPP/XLP-related liver disease are needed.

### BRIEF SUMMARY

Certain embodiments of the invention provide a method of reducing protoporphyrin-IX (PP-IX) level in a subject in need of (e.g., a subject having 1) protoporphyria related hepatobiliary disease, for example, an EPP patient having manifestations of liver disease and/or biliary disease; and/or 2) protoporphyria related skin disease, such as cutaneous photosensitivity, including flare and/or erythema under sunlight exposure, etc.), comprising administering one or more antihistamine (e.g., an H1 receptor antihistamine and/or an H2 receptor antihistamine) to the subject (e.g., for at least 5 or 10 days, 1-2 months, 7 months, or longer).

Certain embodiments of the invention provide a method of reducing protoporphyrin-IX (PP-IX) level in a cell (e.g., hepatocyte, bone marrow cell, or erythrocyte), comprising contacting the cell with one or more antihistamine (e.g., an H1 antihistamine and/or an H2 antihistamine). In certain embodiments, the cell is contacted in vitro. In certain embodiments, the cell is contacted in vivo.

Certain embodiments of the invention provide a method of treating protoporphyria in a subject in need of (e.g., a subject having protoporphyria-related hepatobiliary disease, such as an EPP patient having clinically measurable liver abnormalities), comprising administering one or more antihistamines (e.g., an H1 antihistamine and/or an H2 antihistamine) to the subject.

Certain embodiments of the invention provide a method of treating protoporphyria related hepatobiliary or skin disease in a subject in need of, comprising administering an H1 antihistamine to the subject.

Certain embodiments of the invention provide a method of treating protoporphyria in a subject in need of, comprising administering two or more antihistamines (e.g., an H1 antihistamine and an H2 antihistamine) to the subject.

Certain embodiments of the invention provide a method of treating protoporphyria in a subject in need of, comprising administering chlorcyclizine to the subject.

Certain embodiments of the invention provide a method as described herein.

Certain embodiments of the invention provide one or more antihistamine as described herein for the prophylactic or therapeutic treatment of a proporphyria disease or a proporphyria related hepatobiliary or skin disease in a subject.

Certain embodiments of the invention provide one or more antihistamine as described herein to prepare a medicament for the treatment of a proporphyria disease or a proporphyria related hepatobiliary or skin disease in a subject.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1G. CCZ reduces PP-IX accumulation in experimental EPP. (FIG. 1A) Representative fluorescence images (Texas Red in upper panels, GFP in lower panels) of EPP zebrafish larvae treated with the vehicle DMSO (Control) and chlorcyclizine (CCZ) at the indicated concentrations (0, 2, 5, or 10 μM). (FIG. 1B) Quantification of zebrafish liver PP-IX fluorescence as mean±SD (9±4%, 100±20%, 78±15%, 66±14%, 56±20% at the CCZ concentrations of 0, 2, 5, 10 μM, respectively). (FIG. 1C) PP-IX level in the zebrafish culture medium normalized to the level in the DMSO-treated controls (n≥10 larvae per condition, N=4 experiments). (FIG. 1D) Relative PP-IX fluorescence in the FVB/N mouse strain hepatocytes and culture medium normalized to the level in the untreated ALA+DFO group at 12 h post-treatment with the indicated concentrations of CCZ. Data are shown as mean±SD (N=4 experiments). (FIG. 1E) Representative hematoxylin and eosin (H&E) stained liver sections and quantification of PP-IX area in the ferrochelatase mutant (fch/fch) EPP mice treated with phosphate buffered saline (PBS) or CCZ in PBS. Arrows highlight medium-sized PP-IX deposits (n=8 mice per group, N=2 experiments). Left upper panel (PBS) and right upper panel (CCZ) relate to female mice. Left lower panel (PBS) and right lower panel (CCZ) relate to male mice. (FIG. 1F) Quantification of total PP-IX deposit counts in the fch/fch liver. (FIG. 1G) Quantification of small-, medium-, and large-sized PP-IX deposits. Data are shown as mean±SD (n=8 mice/group, N=2 experiments). Statistical analysis was performed by two-tailed student's t-test, ****P<0.0001, ***P<0.001, **P<0.01, *P<0.05.

FIGS. 2A-2F. CCZ reduces tissue PP-IX levels and liver injury and protein oxidation in female but not male fch/fch mice. (FIG. 2A) PP-IX levels in the liver, erythrocytes, bone marrow, and internal rectal stool. (FIG. 2B) Plasma alanine aminotransferase (ALT), alkaline phosphatase (ALP), and total bilirubin levels. (FIG. 2C) Cage stool was collected every 48 hours. Total stool PP-IX level over the 10-day study period was calculated using the integrated area under the fluorescence curve. Data are shown as mean±SD (n=8-11 mice/group, N=2 experiments). Statistical analysis was performed by ANOVA test, ***P<0.001, **P<0.01, *P<0.05. (FIG. 2D) Immunoblots of the protein p62 monomer and its high molecular weight aggregates in 2% SDS-containing buffer. (FIG. 2E) Densitometry analysis of p62 monomer and high molecular weight aggregates. (FIG. 2F) Oxyblots (as a measure of protein oxidation) of total fch/fch or wild-type livers treated with the vehicle PBS or CCZ. Statistical analysis was performed by two-tailed student's t-test, ***P<0.001, **P<0.01, *P<0.05. CM, Coomassie staining.

FIGS. 3A-3F. Effect of CCZ in DDC-fed mice on PP-IX levels and liver injury, and the role of the histamine pathway in porphyrin-stressed mouse hepatocytes. (FIG. 3A) Representative H&E liver sections and quantification of PP-IX areas in the 3,5-diethoxycarboncyl-1,4-dihydrocollidine (DDC)-fed mice (female and male) treated with PBS or CCZ. Arrows indicate PP-IX deposits. (FIG. 3B) PP-IX levels in the liver and internal (isolated from the intestine) rectal stool in female (F) and male (M) mice. (FIG. 3C) Plasma ALT and ALP levels. (FIG. 3D) Intracellular and medium PP-IX levels following treatment of cultured mouse hepatocytes with histamine, the H1 blocker fexofenadine (FXF), and H2-blockers cimetidine (CMT) and ranitidine (RNT) at the indicated concentrations. (FIG. 3E) CCZ and CMT were tested individually at 10 μM. Intracellular and medium PP-IX levels were measured in the absence of added histamine. (FIG. 3F) CCZ and CMT were tested individually at 10 μM. Intracellular and medium PP-IX levels in the presence of added histamine (HT, 0.5 μM). Data are shown as mean±SD (N≥3 experiments). Statistical analysis was performed by one-way ANOVA test, ***P<0.001, **P<0.01, *P<0.05.

FIGS. 4A-4D. Mast cell presence and HRH1 expression in wild-type and fch/fch livers. (FIG. 4A) May-Grunwald Giemsa staining of wild-type and fch/fch livers. Mast cell staining is highlighted by black arrows. (FIG. 4B) Immunofluorescence staining of mast cell protease 1 (MCP1) in wild-type (WT) and fch/fch livers (MCP1 puncta are indicated by white arrows) with quantified relative staining shown. (FIG. 4C) Immunoblots of the histamine receptor H1 (HRH1) in total liver lysates obtained from fch/fch and WT female and male mice with or without treatment with CCZ. (FIG. 4D) Densitometry analysis of HRH1 expression shown in FIG. 4C. HRH1 expression is suppressed in female fch/fch mice when compared to their WT controls, and CCZ further decreases HRH1 expression preferentially in female livers. Data are shown as mean±SD (n=3 livers/group). Statistical analysis was performed using the one-way ANOVA test, *P<0.05.

FIGS. 5A-5C. CCZ reduces mast cell presence and histamine levels in the female liver and plasma. (FIG. 5A) May-Grunwald Giemsa staining and immunofluorescence staining of mast cell protease 1 (MCP1) on fch/fch and wild-type livers. MCP1 puncta are indicated by white arrows. Expressions of MCP1 puncta are shown as mean percent cell area±SD (n=3 livers, 2 image fields/liver). (FIG. 5B) Immunoblot and densitometry analysis of MCP1 in fch/fch and wild-type livers (n=3 livers/group). CM, Coomassie staining. (FIG. 5C) Histamine levels in the liver and plasma. Data are shown as mean±SD (n=3 mice/group). Statistical analysis was performed by one-way ANOVA test, ****P<0.0001, ***P<0.001, **P<0.01, *P<0.05.

FIGS. 6A-6G. Expression and subcellular partitioning of CAR and FXR. (FIG. 6A) Schematic diagram of the constitutive active/androstane receptor/retinoid X receptor (CAR/RXR) and farnesoid X receptor (FXR/RXR) gene transactivation system. (FIG. 6B) Relative mRNA expressions of Nrli3/CAR and Nr1h4/FXR in female and male fch/fch versus wild-type (WT) livers (+/−CCZ). In WT male livers, CCZ treatment decreased CAR gene expression. (FIG. 6C) CAR and FXR protein expression is suppressed in fch/fch liver. CCZ administration decreases CAR and FXR protein expression further in livers of female fch/fch mice. (FIG. 6D) Liver homogenates were separated into nuclear- and cytoplasmic-enriched fractions. Baseline CAR and FXR expression is comparable in female and male fch/fch livers. Lamin B1 is used as a nuclear marker. CM=Coomassie staining to show equal protein loading. (FIG. 6E) CCZ enriches nuclear localization of CAR, RXR, and FXR in female fch/fch livers. (FIG. 6F) Relative protein expression of multidrug resistance protein 4 (MRP4) and bile salt export pump (BSEP). The MRP4 monomer is indicated by arrowhead. Higher molecular weight aggregates of MRP4 are highlighted by white dotted boxes. (FIG. 6G) Relative mRNA expression of MRP4 (upper row) and BSEP (lower row) in livers of female and male fch/fch (−/+CCZ) and WT mice. Statistical analysis was performed using the one-way ANOVA test. ***P<0.001, **P<0.01, *P<0.05.

FIG. 7. Schematic diagram of CCZ and histamine action in experimental EPP. In fch/fch liver, mast cell presence and histamine level increase. CCZ treatment depletes hepatic MC infiltrates and histamine levels. Histamine receptor types-1/2 (H1R/H2R) are expressed in human and mouse hepatocytes. When tested in porphyrin-stressed hepatocytes, histamine further increases intracellular PP-IX levels and induces protein aggregation (left hepatocyte/orange). Administration of HIR blocker CCZ or H2R blocker CMT reverses this effect, including inhibition of PP-IX-medicated protein aggregation (right hepatocyte/blue). CAR (constitutive androstane receptor) and FXR (farnesoid X receptor) were found to translocate into the nucleus and transactivate bile acid transporters BSEP (bile salt export pump) and MRP4 (multidrug resistance protein-4) expression, leading to enhanced PP-IX excretion (middle hepatocyte/blue).

FIG. 8. Schematic of the high-throughput drug screening in experimental EPP zebrafish. 2,560 compounds were multiplexed into 640 test wells. Positive hits were identified on the basis of significant reduction of the PP-IX (red) signal in EPP zebrafish livers (n≥10 fish larvae, N≥4 experiments). Funnel cartoons represent the progressive flow in the number of active compounds toward a validated optimal hit/compound for further investigation. Note the decrease in red liver fluorescence (right upper part of the schematic).

FIGS. 9A-9C. Liver-to-body weight (LBW) ratio and tissue PP-IX levels in fch/fch mice. (FIG. 9A) fch/fch mice manifest increased % liver-to-body weight ratio that is nearly double the ratio of wild-type (WT) mice (female fch: 9.3±0.8%, female WT: 4.8±0.1%; male fch: 10±0.9%, male WT: 5.1±0.3%). CCZ does not alter the LBW ratio. Data are shown as mean±SD (n=5-8 mice per group, N=2 experiments). Statistical analysis was performed using the one-way ANOVA test, ****P<0.0001. (FIG. 9B) PP-IX levels in plasma and bile obtained from the indicated mouse genotypes (F, female; M, male) treated with PBS (i.e., vehicle, Veh) or CCZ (dissolved in PBS). (FIG. 9C) Stool was collected every 48 h from the mouse cages housing female and male fch/fch mice that were administered PBS or CCZ. Stool PP-IX levels were then measured. Data are shown as mean±SD (n=8-11 mice per group, N=2 experiments). Statistical analysis was performed using the ANOVA test. ***P<0.001, **P<0.01, *P<0.05.

FIG. 10. p62 immune staining of liver sections from fch/fch mice treated with vehicle (PBS) or CCZ. Representative images of liver sections isolated from female and male fch/fch mice, that were administered PBS or CCZ, were stained with 4′,6-diamidino-2-phenylindole (DAPI, to label nuclei) and anti-p62 antibody. Female but not male livers showed reduced p62 staining (displayed in pink).

FIGS. 11A-11C. Liver-to-body weight (Lv/Bw) ratio and PP-IX counts in DDC livers. (FIG. 11A) Prominent Lv/Bw was observed in mice fed with DDC-containing diet for 5 days [female DDC: 7.2±0.4%; female normal diet (ND): 4.7±0.6%; male DDC: 8.7±0.9%; male ND: 5.0±0.1%]. CCZ further increases the liver-to-body weight ratio in the DDC-fed female mice (female DDC+CCZ: 9.0±0.8%). Data are shown as mean±SD (n=2-10 mice per group, N=2). Statistical analysis was performed using the one-way ANOVA test. ****P<0.0001; **P<0.01. (FIG. 11B) Quantification of total PP-IX deposits in livers isolated from DDC-fed (5 days) mice. (FIG. 11C) Quantification of small-, medium-, and large-sized PP-IX deposits in the livers of DDC-fed mice. For FIG. 11B and FIG. 11C, data are shown as mean±SD (n=5 mice per group, N=2). Statistical analysis was performed using the one-way ANOVA test. ****p<0.0001, *P<0.05.

FIGS. 12A-12B. Gene and protein expressions of heme biosynthesis enzymes. (FIG. 12A) ALAS1, ALAD, HMBS, PPOX, and FECH porphyrin biosynthetic enzyme gene expressions are suppressed in the female fch/fch liver. ALAS1, UROD, and FECH porphyrin biosynthetic enzyme gene expressions are suppressed in the male fch/fch liver. (FIG. 12B) ALAS1, FECH, ALAD, and UROD protein expression are suppressed in the fch/fch livers. Under the experimental conditions used in this study, high molecular weight (HMW) aggregates of ALAD and UROD (and not ALAS1 or FECH) were detected in the fch/fch livers. There is an upward shift in ALAD and UROD monomers as indicated by the arrowheads. Data are shown as mean±SD (n=2-3 livers per group). Statistical analysis was performed by one-way ANOVA test, ***P<0.001. **P<0.01. *P<0.05.

FIG. 13. MRP4 and BSEP immunostaining in PBS- and CCZ-treated fch/fch livers. Representative images of liver sections stained with DAPI (to label nuclei) and anti-MRP4 and anti-BSEP antibodies. The MRP4 (pink) and BSEP (yellow) signal increases in the female livers following CCZ treatment. Asterisks indicate large bile ducts. White arrows indicate apical BSEP transporters that appear as yellow lines.

FIG. 14. H1 antihistamine safety recommendations for porphyria patients. Data were accessed from the American Porphyria Foundation (APF) database and the Norwegian Porphyria Centre (NAPOS) database. Color key: dark green=safe; light green=probably safe; yellow=probably unsafe; red=unsafe.

FIGS. 15A-15C. Effect of the second generation H1 antihistamines, fexofenadine and cetirizine, on protein aggregation and porphyrin accumulation in livers of fch/fch mice. (FIG. 15A) Male fch mice were treated daily with fexofenadine (FXD, 10 mg/kg mouse weight in DMSO), cetirizine (CET, 20 mg/kg mouse weight in phosphate buffered saline, PBS, pH7.4). In addition, normal wildtype (WT) mice (same genotype, BALB/c, as the fch mice) were treated with vehicle (PBS). All treatments were by daily intraperitoneal administration for 5 days (4 mice were used for each experimental condition for FXD, and 2 mice per experimental condition for CET). On day 6, livers were isolated followed by immunoblot analysis of the total liver extracts using anti-p62 antibody. Protein concentrations were measured using the BCA assay (Pierce™), and equal amount of protein was analyzed from each liver. Coomassie (CM) staining of the liver lysates isolated from the fch/fch livers used for immunoblotting is included to show equal protein loading. (FIG. 15B) Densitometry analysis of the highlighted protein band intensities are shown. (FIG. 15C) PP-IX levels in the total cell lysate of fch/fch mouse livers (normalized to the protein concentrations) are shown. Data is presented as mean±SD. Statistical analysis was performed by ANOVA testing, ****P<0.0001, ***P<0.001, **P<0.01, *P<0.05.

FIGS. 16A-16C. Effect of the H2 antihistamine, cimetidine, on protein aggregation and porphyrin accumulation in livers of fch/fch mice. (FIG. 16A) Male fch mice were treated daily for 5 days with cimetidine (CMT, 20 mg/kg mouse weight in PBS) or vehicle (4 mice per group). Two WT mice were used as control. On day 6, livers were isolated followed by immunoblot analysis of the total liver extracts using anti-p62 antibody. Coomassie (CM) staining of the total cell lysates used for immunoblotting is included to show equal protein loading. (FIG. 16B) Densitometry analysis of the highlighted protein band intensities analyzed by immunoblotting are shown. (FIG. 16C) PP-IX levels in the total cell lysate of the fch/fch mouse livers (normalized to the protein concentrations) are shown. Data are shown as mean±SD. Statistical analysis was performed by ANOVA testing; *P<0.05.

FIGS. 17A-17D. Mouse proteins aggregate in skin samples from ears of fch/fch mice when analyzed using non-reducing SDS-gel electrophoresis conditions, and the effect of cetirizine on skin protein aggregation and porphyrin levels. (FIG. 17A) Immunoblots of the epidermal keratins 5, 14, and 10 (K5, K14, and K10) and collagen VI (Col VI) in skin extracts isolated from the ears of wildtype and fch mice. Note the marked formation of high molecular weight (hmw) protein aggregates in fch ear skin when analyzed under non-reducing conditions as compared with reducing conditions (i.e., in the presence of the reducing agent, beta-mercaptoethanol). The Col VI is shown under reducing conditions. (FIG. 17B) fch mice were treated daily with vehicle or CET as in FIG. 15 followed by analysis of ear total protein homogenates by immunoblotting of the indicated proteins (K5, K14, K10, Col I a2) after separation under nonreducing conditions to focus on the aggregate formation. CM staining of the analyzed skin proteins is included to show equal protein loading. (FIG. 17C) Note the loss of the uppermost hmw protein species (arrowheads in panel of FIG. 17B) in K5, and the relative decrease in K10, K14, and Col I a2 skin proteins under non-reducing conditions. (FIG. 17D) Porphyrin levels are shown in the skin samples isolated from wildtype, fch, and fch mice treated with CET.

FIGS. 18A-18C. The effect of cimetidine on skin protein aggregation and porphyrin levels. (FIG. 18A) fch male mice were treated with cimetidine or vehicle (CMT, 20 mg/kg mouse weight, in PBS; 4 mice per condition) daily for 5 days. Four mice were also given vehicle by injection daily for 5 days. On day 6, the skin ears were collected from the 12 mice followed by solubilization in buffer containing 5% SDS and 8M urea. The ear protein extracts were analyzed under nonreducing conditions (except for the two collagen proteins which were analyzed under reducing conditions) by immunoblotting using antibodies to K5, K14, and K10 to assess alterations in the monomer and high molecular weight (hmw) oxidized forms. Also shown are the blots of the two collagen proteins (Col I a2, and Col VI) under reducing conditions. Two of the 4 skins extracts from wildtype (WT) ears were randomly selected for immunoblot analysis. (FIG. 18B) Densitometric scanning of the monomer band intensities and the proteins analyzed by immunoblotting in FIG. 18B. (FIG. 18C) Skin porphyrin levels are shown in the skin samples isolated from the 12 wildtype, fech, and fech mice treated with CMT. Data are shown as mean±SD. Statistical analysis was performed by ANOVA testing; ***P<0.001, ****P<0.0001.

### DETAILED DESCRIPTION

Porphyrias are genetic disorders that arise from defects in heme production, which cause build-up of intermediates called porphyrins. In the disorder erythropoietic protoporphyria, skin disease (e.g., cutaneous photosensitivity) is the most common occurrence among EPP patients. In addition, liver is commonly a target of excess porphyrin accumulation that can lead to liver failure. Bone marrow and liver transplantation are currently the only treatment options available for patients with EPP and decompensating liver disease.

To address this major unmet medical need, as described herein and also in Example 1, we screened 2560 compounds and identified chlorcyclizine, a H1 antihistamine traditionally used to treat allergy, as an unexpected drug that markedly reduces porphyrin accumulation and liver injury. We show herein that histamine itself is porphyrinogenic and worsens porphyrin accumulation, and its blockade by several antihistamine drug reverses porphyrin accumulation. Our findings suggest that targeting the histamine pathway with antihistamines could ameliorate the development or progression of liver disease in patients with erythropoietic protoporphyria. For example, the repurposing of chlorcyclizine and other antihistamines as protoporphyrin-IX (PP-IX) lowering agents could reduce PP-IX accumulation and liver injury (e.g., that is caused by the oxidation and PP-IX mediated aggregation of hepatocyte proteins), increase stool PP-IX excretion, and therefore could serve as safe and effective alternatives to invasive surgery.

Accordingly, certain embodiments of the invention provide a method of reducing PP-IX level or treating protoporphyria in a subject (e.g., a subject having: 1) protoporphyria related hepatobiliary disease, such as an EPP patient having manifestations of liver and/or biliary disease; and/or 2) protoporphyria related skin disease, such as cutaneous photosensitivity, including flare and/or erythema after sunlight exposure, etc.) in need of, comprising administering one or more antihistamine (e.g., an H1 antihistamine and/or an H2 antihistamine) to the subject.

In certain embodiments, the one or more antihistamine comprises an H1 antihistamine.

In certain embodiments, the one or more antihistamine comprises an H2 antihistamine.

In certain embodiments, the one or more antihistamine comprises an H1 antihistamine described herein and an H2 antihistamine described herein.

In certain embodiments, the one or more antihistamine does not comprise an H2 antihistamine.

In certain embodiments, the subject has protoporphyria related hepatobiliary disease. In certain embodiments, the subject has protoporphyria related liver disease (e.g., having serological evidence of liver injury). In certain embodiments, the subject has protoporphyria related biliary disease (e.g., gallstones).

In certain embodiments, the subject has protoporphyria related skin disease (e.g., cutaneous photosensitivity, including flare and/or erythema).

In certain embodiments, the method is a method of reducing PP-IX level or treating protoporphyria in a subject in need of, comprising administering two or more antihistamines (e.g., a H1 antihistamine as described herein and a H2 antihistamine as described herein).

In certain embodiments, the method is a method of reducing PP-IX level or treating protoporphyria related hepatobiliary disease in a subject in need of, comprising administering a H1 antihistamine, and optionally further comprises administering a H2 antihistamine.

In certain embodiments, the method is a method of reducing PP-IX level or treating protoporphyria related skin disease in a subject in need of, comprising administering an H1 antihistamine, and optionally further comprises administering an H2 antihistamine.

In certain embodiments, the method is a method of reducing PP-IX level or treating protoporphyria related hepatobiliary disease in a subject in need of, comprising administering a H2 antihistamine, and optionally further comprises administering a H1 antihistamine.

In certain embodiments, the method is a method of reducing PP-IX level or treating protoporphyria related skin disease in a subject in need of, comprising administering an H2 antihistamine, and optionally further comprises administering an H1 antihistamine.

As used herein, the term “antihistamine” refers to an inhibitor agent or a salt thereof that inhibits the activity of a histamine receptor. For example, the histamine receptor mediated signaling activity can be reduced or blocked by the inhibitor. In certain embodiments, the binding between histamine and a histamine receptor is reduced or blocked by the inhibitor. In certain embodiments, the antihistamine is H1 antihistamine. In certain embodiments, the antihistamine is H2 antihistamine.

As used herein, the term “H1 antihistamine” or “H1 receptor antihistamine” are used interchangeably and refers to an inhibitor agent or a salt thereof that inhibits the activity of histamine receptor H1 (HRH1). H1 antihistamines (e.g., clinically approved H1 antihistamines) are known in the art and described herein. For illustrative purpose, as non-limiting examples, in certain embodiments, an H1 antihistamine is selected from the group consisting of Hydroxyzine, Mepyramine, Tripelennamine, Cetirizine, Fexofenadine, Levocetirizine, Loratadine, Brompheniramine, Chlorpheniramine, Dimenhydrinate, diphenhydramine, Doxylamine, and Chlorcyclizine. In certain embodiments, an H1 antihistamine is selected from the group consisting of Cetirizine, Fexofenadine, Levocetirizine, Loratadine, Acrivastine, Bepotastine, Bilastine, Desloratadine, Ebastine, Fexofenadine, Ketotifen, Mizolastine, Quifenadine, Rupatadine, and Terfenadine. In certain embodiments, an H1 antihistamine is selected from the group consisting of Cetirizine, Fexofenadine, Levocetirizine, and Loratadine. In certain embodiments, an H1 antihistamine is selected from the group consisting of Cetirizine, Chlorcyclizine, Cyclizine, Levocetirizine, Cyproheptadine, Desloratadine, Ebastine, Fexofenadine, Terfenadine, Promethazine, Thiethylperazine, Chlorpheniramine, and Mizolastine. In certain embodiments, an H1 antihistamine is a second generation H1 antihistamine, such as Cetirizine, or Fexofenadine.

As used herein, the term “H2 antihistamine” or “H2 receptor antihistamine” are used interchangeably and refers to an inhibitor agent or a salt thereof that inhibits the activity of histamine receptor H2 (HRH2). H2 antihistamines (e.g., clinically approved H2 antihistamines) are known in the art and described herein. For illustrative purpose, as non-limiting examples, in certain embodiments, a H2 antihistamine is selected from the group consisting of Cimetidine, Ranitidine, Famotidine, Nizatidine, Tiotidine, Roxatidine, Lafutidine, and Sufotidine. In certain embodiments, a H2 antihistamine is selected from the group consisting of Cimetidine, Lafutidine, Ranitidine, Tiotidine, Famotidine, Nizatidine, and Roxatidine.

As used herein, the term “protoporphyria” refers to erythropoietic protoporphyria (EPP) or X-linked protoporphyria (XLP). In certain embodiments, the subject has EPP. In certain embodiments, the subject has EPP related hepatobiliary disease. In certain embodiments, the subject has XLP. In certain embodiments, the subject has XLP related hepatobiliary disease.

As used herein, the term “protoporphyria related hepatobiliary disease” refers to a clinically measurable liver and/or biliary disease or condition in a subject having erythropoietic protoporphyria (EPP) or X-linked protoporphyria (XLP). XLP also leads to PP-IX accumulation due to a gain of function mutation in the enzyme 5′-aminolevulinate synthase 2 (or erythroid ALA-synthase) that is found in developing erythrocytes. Patients with XLP also develop photosensitivity and hepatobiliary disease. Although for the majority of patients, protoporphyria does not necessarily affect survival, a subset of protoporphyria patient group (e.g., about 10%-25%) may have protoporphyria related liver disease or biliary (e.g., gallbladder stone-related) disease due to enzyme deficiency/defect and the ensuing PP-IX accumulation stress, and/or protein oxidation burden in liver, and PP-IX crystallization in bile that can lead to gallstones. An even smaller subset of such patients may develop from moderate, chronic liver disease to more severe or life-threatening end stage liver disease. In certain embodiments, the subject having protoporphyria related hepatobiliary disease has one or more elevated liver enzymes (e.g., ALP and/or ALT) or total bilirubin in a blood sample. In certain embodiments, the subject has steatosis. In certain embodiments, the subject has liver fibrosis, cirrhosis, increased liver stiffness, and/or gallbladder stones (e.g., as measured by CT, MRI, ultrasound, or elastography). In certain embodiments, the subject has jaundice. In certain embodiments, protoporphyria related hepatobiliary disease is a liver disease. In certain embodiments, protoporphyria related hepatobiliary disease is a biliary disease in gallbladder and/or bile ducts. In certain embodiments, the subject has cholestasis. In certain embodiments, the subject has cholecystitis. In certain embodiments, the subject has gallstone.

As used herein, the term “protoporphyria related skin disease” refers to a clinically measurable cutaneous disease or condition in a subject having erythropoietic protoporphyria (EPP) or X-linked protoporphyria (XLP).

In certain embodiments, a method described herein comprises administering an H1 antihistamine to the subject.

For illustration purpose, as non-limiting examples, in certain embodiments, the H1 antihistamine is selected from the group consisting of Cetirizine, Chlorcyclizine, Cyclizine, Levocetirizine, Cyproheptadine, Desloratadine, Ebastine, Fexofenadine, Terfenadine, Promethazine, Thiethylperazine, Chlorpheniramine, and Mizolastine.

In certain embodiments, the H1 antihistamine is Chlorcyclizine or Cyclizine.

In certain embodiments, the H1 antihistamine is Fexofenadine, or Terfenadine.

In certain embodiments, the H1 antihistamine is not Terfenadine.

In certain embodiments, an H1 antihistamine is Cetirizine, Fexofenadine, Levocetirizine, or Loratadine.

In certain embodiments, the H1 antihistamine is Fexofenadine or Chlorcyclizine.

In certain embodiments, the H1 antihistamine is Fexofenadine.

In certain embodiments, the H1 antihistamine is Chlorcyclizine. In certain embodiments, the H1 antihistamine is Chlorcyclizine, Fexofenadine, or Cetirizine.

Without wanting to be bound by theory, for the subset of proporphyria patients that have related hepatobiliary disease such as a chronic liver disease, in certain embodiments, treatment duration that is longer than a 1, 2, or 3-day temporary use of H1 antihistamine may be needed to provide benefits, such as mid-term, long term, or even lifelong use of H1 antihistamine may be needed to confer sustained protection against proporphyria related hepatobiliary disease. These drugs are commonly used as anti-allergy medications and are deemed safe for longterm use as needed.

In certain embodiments, the H1 antihistamine is administered for at least 5, 6, 7, 8, 9, 10, 11, or 12 days. In certain embodiments, the H1 antihistamine is administered for at least 5 days. In certain embodiments, the H1 antihistamine is administered for at least 10 days.

In certain embodiments, the H1 antihistamine is administered for at least 1, 2, 3, 4, 5, 6, 7, or 8 weeks. In certain embodiments, the H1 antihistamine is administered for about 1, 2, 3, 4, 5, 6, 7, or 8 weeks. In certain embodiments, the H1 antihistamine is administered for about 3, 6, or 9 weeks. In certain embodiments, the H1 antihistamine is administered for about 10 or 11 weeks.

In certain embodiments, the H1 antihistamine is administered for at least 1, 2, 3, 4, 5, 6, 7, 8, 9, 10, 11, or 12 months. In certain embodiments, the H1 antihistamine is administered for about 1, 2, 3, 4, 5, 6, 7, 8, 9, 10, 11, or 12 months. In certain embodiments, the H1 antihistamine is administered for about 3, 6, or 9 months. In certain embodiments, the H1 antihistamine is administered for about 2, or 4 months.

In certain embodiments, the H1 antihistamine is administered for at least 1, 2, 3, 4, 5, 6, 7, or 8 years. In certain embodiments, the H1 antihistamine is administered for about 1, 2, 3, 4, 5, 6, 7, 8, 9, 10 years or longer. In certain embodiments, the H1 antihistamine is administered for about 3 years, or longer.

In certain embodiments, a method described herein does not comprise administering an H2 antihistamine to the subject. In certain embodiments, a method described herein does not comprise administering cimetidine to the subject.

In certain embodiments, a method described herein comprises, or further comprises, administering a H2 antihistamine to the subject.

In certain embodiments, the H2 antihistamine is selected from the group consisting of Cimetidine, Lafutidine, Ranitidine, Tiotidine, Famotidine, Nizatidine, and Roxatidine. In certain embodiments, the H2 antihistamine is Cimetidine or Ranitidine. In certain embodiments, the H2 antihistamine is Cimetidine. In certain embodiments, the H2 antihistamine is not Cimetidine. In certain embodiments, the H2 antihistamine is Ranitidine.

In certain embodiments, the H2 antihistamine is administered for at least 5, 6, 7, 8, 9, 10, 11, or 12 days. In certain embodiments, the H2 antihistamine is administered for at least 5 days. In certain embodiments, the H2 antihistamine is administered for at least 10 days.

In certain embodiments, the H2 antihistamine is administered for at least 1, 2, 3, 4, 5, 6, 7, or 8 weeks. In certain embodiments, the H2 antihistamine is administered for about 1, 2, 3, 4, 5, 6, 7, or 8 weeks. In certain embodiments, the H2 antihistamine is administered for about 3, 6, or 9 weeks. In certain embodiments, the H2 antihistamine is administered for about 10 or 11 weeks.

In certain embodiments, the H2 antihistamine is administered for at least 1, 2, 3, 4, 5, 6, 7, 8, 9, 10, 11, or 12 months. In certain embodiments, the H2 antihistamine is administered for about 1, 2, 3, 4, 5, 6, 7, 8, 9, 10, 11, or 12 months. In certain embodiments, the H2 antihistamine is administered for about 3, 6, or 9 months. In certain embodiments, the H2 antihistamine is administered for about 2, or 4 months.

In certain embodiments, the H2 antihistamine is administered for at least 1, 2, 3, 4, 5, 6, 7, or 8 years. In certain embodiments, the H2 antihistamine is administered for about 1, 2, 3, 4, 5, 6, 7, 8, 9, 10 years or longer. In certain embodiments, the H2 antihistamine is administered for about 3 years, or longer.

In certain embodiments, a method described herein comprises administering two or more antihistamines (e.g., a H1 antihistamine and a H2 antihistamine) to the subject.

In certain embodiments, the antihistamines are administered for at least 5, 6, 7, 8, 9, 10, 11, or 12 days. In certain embodiments, the antihistamines are administered for at least 5 days. In certain embodiments, the antihistamines are administered for at least 10 days.

In certain embodiments, the antihistamines are administered for at least 1, 2, 3, 4, 5, 6, 7, or 8 weeks. In certain embodiments, the antihistamines are administered for about 1, 2, 3, 4, 5, 6, 7, or 8 weeks. In certain embodiments, the antihistamines are administered for about 3, 6, or 9 weeks. In certain embodiments, the antihistamines are administered for about 10 or 11 weeks.

In certain embodiments, the antihistamines are administered for at least 1, 2, 3, 4, 5, 6, 7, 8, 9, 10, 11, or 12 months. In certain embodiments, the antihistamines are administered for about 1, 2, 3, 4, 5, 6, 7, 8, 9, 10, 11, or 12 months. In certain embodiments, the antihistamines are administered for about 3, 6, or 9 months. In certain embodiments, the antihistamines are administered for about 2, or 4 months.

In certain embodiments, the antihistamines are administered for at least 1, 2, 3, 4, 5, 6, 7, or 8 years. In certain embodiments, the antihistamines are administered for about 1, 2, 3, 4, 5, 6, 7, 8, 9, 10 years or longer. In certain embodiments, the antihistamines are administered for about 3 years, or longer.

In certain embodiments, the pathogenic accumulation or generation of excessive protoporphyrin-IX (PP-IX) level in the subject is reduced by a method described herein. In certain embodiments, the accumulation or generation of PP-IX in one or more organs, tissues, and/or cells (e.g., hepatocyte or erythrocyte), or in plasma or serum are reduced. In certain embodiments, PP-IX level in a biological sample (e.g., a blood sample or biopsy sample) from the subject is reduced. In certain embodiments, PP-IX level in a plasma or serum sample from the subject is reduced.

In certain embodiments, PP-IX level is reduced in a hepatocyte. In certain embodiments, PP-IX level in a hepatocyte is reduced by at least 10%, 15%, 20%, 30%, 40%, 50%, 60%, 70%, 80% or more (e.g., as compared to a control, such as vehicle or placebo control, or a control wherein no H1 and/or H2 antihistamine was contacted with the cell, in vitro or in vivo).

In certain embodiments, PP-IX level in liver is reduced in the subject. In certain embodiments, PP-IX level in liver is reduced by at least about 10%, 15%, 20%, 30%, 40%, 50%, 60%, 70%, 80% or more (e.g., as compared to a control, such as vehicle or placebo control, or a control wherein no H1 and/or H2 antihistamine was administered). In certain embodiments, PP-IX level in liver is reduced by at least about 20%, or 30%.

In certain embodiments, PP-IX level in skin is reduced in the subject. In certain embodiments, PP-IX level in skin is reduced by at least 10%, 15%, 20%, 30%, 40%, 50%, 60%, 70%, 80% or more (e.g., as compared to a control, such as vehicle or placebo control, or a control wherein no H1 and/or H2 antihistamine was administered). In certain embodiments, PP-IX level in skin is reduced by at least 20%, or 30%.

In certain embodiments, porphyrin-mediated protein aggregation (e.g., HMW protein formation) or oxidation (e.g., in liver and/or skin) is reduced in the subject. In certain embodiments, porphyrin-mediated protein aggregation or oxidation in liver or skin is reduced by at least 10%, 15%, 20%, 30%, 40%, 50%, 60%, 70%, 80% or more (e.g., as compared to a control, such as vehicle or placebo control, or a control wherein no H1 and/or H2 antihistamine was administered). In certain embodiments, porphyrin-mediated protein aggregation or oxidation in liver or skin is reduced by at least 20%, or 30%.

In certain embodiments, one or more of keratin (e.g., K5, K10, or K14) and/or collagen (e.g., Col VI, Col I a2) protein aggregation (e.g., HMW protein formation) or oxidation in skin is reduced in the subject. In certain embodiments, one or more of keratin (e.g., K5, K10, or K14) and/or collagen (e.g., Col VI, Col I a2) aggregation in skin is reduced by at least 10%, 15%, 20%, 30%, 40%, 50%, 60%, 70%, 80% or more (e.g., as compared to a control, such as vehicle or placebo control, or a control wherein no H1 and/or H2 antihistamine was administered). In certain embodiments, one or more of keratin (e.g., K5, K10, or K14) and/or collagen (e.g., Col VI, Col I a2) aggregation in skin is reduced by at least 20%, or 30%.

In certain embodiments, PP-IX level is reduced in an erythrocyte, or precursor thereof in bone marrow. In certain embodiments, PP-IX level in an erythrocyte or precursor thereof is reduced by at least about 10%, 15%, 20%, 30%, 40%, 50%, 60%, 70%, 80% or more (e.g., as compared to a control, such as vehicle or placebo control, or a control wherein no H1 and/or H2 antihistamine was contacted with the cell, in vitro or in vivo).

In certain embodiments, PP-IX level in circulating erythrocytes from a blood sample of the subject is reduced. In certain embodiments, PP-IX level in circulating erythrocytes is reduced by at least about 10%, 15%, 20%, 30%, 40%, 50%, 60%, 70%, 80% or more (e.g., as compared to a control, such as vehicle or placebo control, or a control wherein no H1 and/or H2 antihistamine was administered). In certain embodiments, PP-IX level in circulating erythrocytes is reduced by at least about 20%, or 30%.

In certain embodiments, PP-IX level in bone marrow is reduced. In certain embodiments, PP-IX level in bone marrow is reduced by at least about 10%, 15%, 20%, 30%, 40%, 50%, 60%, 70%, 80% or more (e.g., as compared to a control, such as vehicle or placebo control, or a control wherein no H1 and/or H2 antihistamine was administered). In certain embodiments, PP-IX level in bone marrow is reduced by at least about 20%, or 30%.

In certain embodiments, PP-IX level in a plasma or serum sample of the subject is reduced. In certain embodiments, PP-IX level in a plasma or serum sample of the subject is reduced by at least about 10%, 15%, 20%, 30%, 40%, 50%, 60%, 70%, 80% or more (e.g., as compared to a control, such as vehicle or placebo control, or a control wherein no H1 and/or H2 antihistamine was administered). In certain embodiments, PP-IX level in a plasma or serum sample of the subject is reduced by at least about 20%, or 30%.

In certain embodiments, PP-IX excretion or clearance is enhanced. In certain embodiments, PP-IX level in stool sample of the subject is increased. For example, in certain embodiments, PP-IX level in stool sample of the subject is increased by at least about 30%, 40%, 50%, 60%, 70%, 80%, 90%, 100%, 110%, 120%, 130%, 140%, 150%, 160%, 170%, 180%, 190%, 200%, 220%, 240%, 260%, or more (e.g., as compared to a control, such as vehicle or placebo control, or a control wherein no H1 and/or H2 antihistamine was administered). In certain embodiments, PP-IX level in stool sample of the subject is increased by at least about 50%, 100%, 150%, or 200%.

In certain embodiments, hepatobiliary injury (e.g., liver injury) of the subject having proporphyria related hepatobiliary disease is reduced. In certain embodiments, plasma or serum levels of alanine aminotransferase (ALT), alkaline phosphatase (ALP), and/or total bilirubin are reduced (e.g., previously elevated level returns to within the normal reference range after or during treatment as described herein).

In certain embodiments, the plasma level of ALT is reduced. In certain embodiments, plasma level of ALT is reduced by at least about 10%, 15%, 20%, 30%, 40%, 50%, 60%, 70%, 80% or more (e.g., as compared to a control, such as vehicle or placebo control, or a control wherein no H1 and/or H2 antihistamine was administered).

In certain embodiments, the plasma level of ALP is reduced. In certain embodiments, plasma level of ALP is reduced by at least about 10%, 15%, 20%, 30%, 40%, 50%, 60%, 70%, 80% or more (e.g., as compared to a control, such as vehicle or placebo control, or a control wherein no H1 and/or H2 antihistamine was administered).

In certain embodiments, the total bilirubin level is reduced. In certain embodiments, the total bilirubin level is reduced by at least about 10%, 15%, 20%, 30%, 40%, 50%, 60%, 70%, 80% or more (e.g., as compared to a control, such as vehicle or placebo control, or a control wherein no H1 and/or H2 antihistamine was administered).

In certain embodiments, the histamine level (e.g., in a plasma sample, or in liver) is reduced. In certain embodiments, the histamine level is reduced by at least about 10%, 15%, 20%, 30%, 40%, 50%, 60%, 70%, 80% or more (e.g., as compared to a control, such as vehicle or placebo control, or a control wherein no H1 and/or H2 antihistamine was administered).

In certain embodiments, mast cell number in liver is reduced. In certain embodiments, mast cell number in liver is reduced by at least about 10%, 15%, 20%, 30%, 40%, 50%, 60%, 70%, 80% or more (e.g., as compared to a control, such as vehicle or placebo control, or a control wherein no H1 and/or H2 antihistamine was administered).

In certain embodiments, the method further comprises identifying a subject as having proporphyria related skin disease prior to administering the one or more antihistamine (e.g., an H1 antihistamine and/or an H2 antihistamine) to the subject.

In certain embodiments, the method further comprises identifying a subject as having proporphyria related hepatobiliary disease prior to administering the one or more antihistamine (e.g., a H1 antihistamine and/or a H2 antihistamine) to the subject. In certain embodiments, the identifying comprises determining the liver function of a subject (e.g., imaging liver, or assaying a blood sample, such as a plasma or serum sample, based on one or more markers that may indicate the dysfunction of liver). In certain embodiments, the identifying comprises determining the subject as having elevated liver enzyme(s) in a blood sample (e.g., plasma or serum). In certain embodiments, the subject having protoporphyria related hepatobiliary disease has one or more elevated liver enzymes (e.g., ALP and/or ALT) in a blood sample. In certain embodiments, the subject has an elevated liver enzyme level that is 110%, 120%, 130%, 140%, 150%, 160%, 170%, 180%, 190%, 200% of the upper limit of normal reference range of the enzyme (e.g., ALP or ALT). In certain embodiments, the subject has an elevated liver enzyme level that is equal to or greater than 2 times the upper limit of normal reference range of the enzyme (e.g., ALP or ALT).

In certain embodiments, the identifying comprises obtaining an image of liver and/or gallbladder (e.g., ultrasound, CT, MRI, or elastography).

In certain embodiments, the identifying comprises identifying the subject as having one or more disease or conditions selected from the group consisting of steatosis, cirrhosis, liver stiffness, jaundice, cholestasis, cholecystitis, and gallstone prior to administering the one or more antihistamine (e.g., H1 antihistamine and/or H2 antihistamine).

In certain embodiments, the identifying optionally further comprises determining the subject as a candidate who could benefit from administration of one or more antihistamine (e.g., an H1 antihistamine and/or an H2 antihistamine).

In certain embodiments, the subject is a female. In certain embodiments, the subject is a male.

In certain embodiments, one or more cutaneous photosensitivity symptoms of the subject is reduced. In certain embodiments, flare reaction under blue light or sunlight is reduced. In certain embodiments, erythema under blue light or sunlight is reduced.

In certain embodiments, the method described herein may comprise administering one or more additional non-antihistamine therapeutic agents to the subject. In certain embodiments, the one or more additional non-antihistamine therapeutic agents comprise beta-carotene, oral iron, biopertin, afamelanotide, dersimelagon, or cholestipol.

In certain embodiments, the method described herein does not comprise administering an additional non-antihistamine therapeutic agent to the subject. For example, in certain embodiments, the method described herein does not comprise administering beta-carotene to the subject.

Frequency for administration of H1 and/or H2 antihistamine(s) can range from multiple doses to a single dose per day. In certain embodiments, the H1 and/or H2 antihistamine(s) may be administered, e.g., orally, to a mammal in need of, for example, about once, twice, trice, or four times per day. In certain embodiments, the H1 and/or H2 antihistamine(s) may be administered 7, 8, 9, 10, 11, 12, 13, 14, 15, 16, 17, 18, 19, 20, 21, or more times per week.

In certain embodiments, the H1 or H2 antihistamine dose can range from about 1 mg/kg (body weight) to about 25 mg/kg, from about 1 mg/kg to about 16 mg/kg, from about 1 mg/kg to about 10 mg/kg, from about 2 mg/kg to about 5 mg/kg, from about 1 mg/kg to about 3 mg/kg, or from about 1 mg/kg to about 2 mg/kg.

The amount of the present invention required for use in treatment will vary with the route of administration, the nature of the condition being treated and the age and condition of the patient and will be ultimately at the discretion of the attendant physician or clinician or pharmacist.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological or pathological change or disorder. For example, beneficial or desired clinical results include, but are not limited to, alleviation of symptoms, diminishment of extent of disease or disorder, stabilized (e.g., not worsening) state of disease or disorder, delay or slowing of disease progression, amelioration or palliation of the disease state or disorder, and remission (whether partial or total), whether detectable or undetectable. Those in need of treatment include those already with the disease or disorder as well as those prone to have the disease or disorder or those in which the disease or disorder manifestations are to be prevented. In one embodiment “treat”, “treatment”, or “treating” does not include preventing or prevention.

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

For in vivo use, active ingredient(s) of the invention is generally incorporated into a pharmaceutical composition prior to administration. Within such composition(s), in certain embodiments, one or two antihistamine(s) may be present as active ingredient(s) (i.e., are present at levels sufficient to provide a statistically significant effect on the symptoms of a relevant disease or result in a decrease in porphyrin levels).

Certain embodiments of the invention provide a composition comprising two or more antihistamines as described herein. In certain embodiments, the composition (e.g., a single solid dosage form such as a tablet, or a single liquid dosage form such as a solution) comprises a H1 antihistamine described herein (e.g., chlorcyclizine, or fexofenadine) and a H2 antihistamine described herein (e.g., cimetidine, or ranitidine). In certain embodiments, the composition (e.g., a single tablet or a solution) comprises chlorcyclizine and cimetidine.

A pharmaceutical composition comprises one or more active ingredient(s) in combination with any pharmaceutically acceptable carrier(s) known to those skilled in the art to be suitable for the particular mode of administration. In addition, other pharmaceutically active ingredients (including other therapeutic agents) may, but need not, be present within the composition.

In certain embodiments, the composition is in solid dosage form (e.g., a tablet).

In certain embodiments, the composition is in liquid dosage form.

In certain embodiments, the composition is an aqueous solution.

In certain embodiments, the composition is an emulsion.

The composition(s) or active ingredient(s) (e.g., H1 and/or H2 antihistamine) may be administered, e.g., orally, topically, intranasally, intravenously, subcutaneously, intradermally, intramuscularly, or intraperitoneally. Solutions of the composition or active ingredient(s) of the invention may be prepared in water, saline buffer, optionally mixed with a nontoxic surfactant. Dispersions can also be prepared in glycerol, liquid polyethylene glycols, triacetin, and mixtures thereof and in oils. Under ordinary conditions of storage and use, these preparations may contain a preservative to prevent the growth of microorganisms.

Certain embodiments of the invention also provide a kit comprising one or more composition(s) comprising one or more antihistamine(s) as described herein, packaging material, and instructions for administering the composition(s) (e.g., a H1 antihistamine composition such as a tablet and a H2 antihistamine composition such as a tablet) to a subject for the treatment of proporphyria or related hepatobiliary disease.

In certain embodiments, an H1 antihistamine described herein (e.g., CCZ, FXD, or CET) and an H2 antihistamine described herein (e.g., CMT) are present in two separate compositions (e.g., two separate tablets, two separate topical creams, or one tablet and one topical cream).

In certain embodiments, an H1 antihistamine described herein (e.g., CCZ, FXD, or CET) and an H2 antihistamine described herein (e.g., CMT) are present in a single composition (e.g., a single tablet or a solution).

The composition(s) or active ingredient(s) (e.g., H1 and/or H2 antihistamine) can be formulated according to standard methods (see, for example, Remington's Pharmaceutical Science, latest edition, Mark Publishing Company, Easton, U.S.A), and may comprise pharmaceutically acceptable carriers and/or additives. Exemplary carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed in the present invention are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and amino acids such as glycine, glutamine, asparagine, arginine, and lysine. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50).

In certain embodiments, the composition(s) or active ingredient(s) (e.g., H1 and/or H2 antihistamine) may be systemically administered, e.g., intravenously, subcutaneously, intradermally, orally, in combination with a pharmaceutically acceptable vehicle such as an inert diluent or an assimilable edible carrier. They may be freeze-dried into lyophilized formulation (e.g., lyophilized cake), may be formulated or reconstituted as a liquid dosage form, may be enclosed in hard or soft shell gelatin capsules, may be compressed into tablets, or may be incorporated directly with the food of the patient's diet. For oral therapeutic administration, active ingredient(s) of the invention may be combined with one or more excipients and used in the form of ingestible tablets, buccal tablets, troches, capsules, elixirs, suspensions, syrups, wafers, and the like. Such compositions and preparations should contain at least 0.1% of active ingredient(s) of the present invention. The percentage of the compositions and preparations may, of course, be varied and may conveniently be between about 2 to about 60% of the weight of a given unit dosage form. The amount of the active ingredient(s) of the invention in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed.

For topical administration, the present composition or active ingredient(s) of the invention may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present inhibitor can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

“Systemic delivery,” as used herein, refers to delivery that leads to a broad biodistribution within an organism. Some techniques of administration can lead to the systemic delivery of certain agents, but not others. Systemic delivery means that a useful, preferably therapeutic and nontoxic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding or uptake) before reaching a disease site distal to the site of administration. Systemic delivery can be by any means known in the art including, for example, intravenous, oral, subcutaneous, and intraperitoneal.

“Local delivery,” as used herein, refers to delivery directly to a target site within an organism, e.g., to a localized area of skin using a cream or ointment formulation using a formulation for topical administration.

For in vivo administration, administration can be in any manner known in the art, e.g., by injection, oral administration, inhalation (e.g., intransal or intratracheal), transdermal application, or rectal administration. Administration can be accomplished via single or divided doses. The pharmaceutical compositions can be administered parenterally, i.e., intravenously, intraperitoneally, subcutaneously, or intramuscularly. In some embodiments, the pharmaceutical compositions are administered intravenously or intraperitoneally by a bolus injection (see, e.g., U.S. Pat. No. 5,286,634). The compounds and compositions can be administered by direct injection at the site of disease or by injection at a site distal from the site of disease (see, e.g., Culver, HUMAN GENE THERAPY, Mary Ann Liebert, Inc., Publishers, New York. pp. 70-71 (1994)). The disclosures of the above-described references are herein incorporated by reference in their entirety for all purposes.

In certain embodiments, the pharmaceutical compositions may be delivered by intranasal sprays, inhalation, and/or other aerosol delivery vehicles. Methods for delivering nucleic acid compositions directly to the lungs via nasal aerosol sprays have been described, e.g., in U.S. Pat. Nos. 5,756,353 and 5,804,212. Likewise, the delivery of drugs using intranasal microparticle resins and lysophosphatidyl-glycerol compounds (U.S. Pat. No. 5,725,871) are also well-known in the pharmaceutical arts. Similarly, transmucosal drug delivery in the form of a polytetrafluoroetheylene support matrix is described in U.S. Pat. No. 5,780,045. The disclosures of the above-described patents are herein incorporated by reference in their entirety for all purposes.

The methods of the present invention may be practiced in a variety of hosts. Preferred hosts include mammalian species, such as primates (e.g., humans and chimpanzees as well as other nonhuman primates), canines, felines, equines, bovines, ovines, caprines, rodents (e.g., rats and mice), lagomorphs, and swine.

The term “animal” includes mammalian species, such as a human, mouse, rat, dog, cat, hamster, guinea pig, rabbit, livestock, and the like.

The term “salts” includes any anionic and cationic complex. Non-limiting examples of anions include inorganic and organic anions, e.g., hydride, fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

### CERTAIN EMBODIMENTS OF THE INVENTION

Embodiment 1. A method of treating protoporphyria related hepatobiliary disease in a subject in need of, comprising administering an H1 antihistamine to the subject.

Embodiment 2. The method of Embodiment 1, wherein the H1 antihistamine is administered for at least 5 days.

Embodiment 3. The method of any one of Embodiments 1-2, wherein the H1 antihistamine is administered for at least 10 days.

Embodiment 4. The method of any one of Embodiments 1-3, further comprising administering an H2 antihistamine.

Embodiment 5. The method of any one of Embodiments 1-4, wherein protoporphyria related hepatobiliary injury of the subject is reduced.

Embodiment 6. The method of any one of Embodiments 1-5, wherein protoporphyrin-IX (PP-IX) level in liver, serum, plasma, or red blood cells is reduced.

Embodiment 7. The method of any one of Embodiments 1-6, wherein plasma or serum levels of alanine aminotransferase (ALT), alkaline phosphatase (ALP), and total bilirubin are reduced.

Embodiment 8. The method of any one of Embodiments 1-7, wherein PP-IX level in circulating erythrocytes is reduced.

Embodiment 9. The method of any one of Embodiments 1-8, wherein PP-IX level in stool is increased.

Embodiment 10. The method of any one of Embodiments 1-9, wherein the H1 antihistamine is chlorcyclizine (CCZ), fexofenadine (FXF), or cetirizine (CET).

Embodiment 11. The method of any one of Embodiments 1-10, wherein the H1 antihistamine is chlorcyclizine (CCZ).

Embodiment 12. The method of any one of Embodiments 1-11, further comprising identifying the subject as having protoporphyria related hepatobiliary disease prior to administering the H1 antihistamine to the subject.

Embodiment 13. A method of treating protoporphyria in a subject in need of, comprising administering two or more antihistamines to the subject.

Embodiment 14. The method of Embodiment 13, wherein the two or more antihistamines comprise an H1 antihistamine.

Embodiment 15. The method of any one of Embodiments 13-14, wherein the two or more antihistamines comprise an H1 antihistamine (e.g., chlorcyclizine, fexofenadine, or cetirizine) and an H2 antihistamine (e.g., cimetidine or ranitidine).

Embodiment 16. The method of any one of Embodiments 13-15, wherein the antihistamines are administered for at least 5 days.

Embodiment 17. The method of any one of Embodiments 13-16, wherein the antihistamines are administered for at least 10 days.

Embodiment 18. The method of any one of Embodiments 13-17, wherein the subject has protoporphyria related hepatobiliary disease.

Embodiment 19. The method of claim 18, wherein protoporphyria related hepatobiliary injury of the subject is reduced.

Embodiment 20. The method of any one of Embodiments 18-19, wherein protoporphyrin-IX (PP-IX) level in liver is reduced.

Embodiment 21. The method of any one of Embodiments 18-20, wherein plasma or serum levels of alanine aminotransferase (ALT), alkaline phosphatase (ALP), and total bilirubin are reduced.

Embodiment 22. The method of any one of Embodiments 13-21, wherein PP-IX level in circulating erythrocytes, serum or plasma is reduced.

Embodiment 23. The method of any one of Embodiments 13-21, wherein PP-IX level in stool is increased.

Embodiment 24. The method of any one of Embodiments 14-23, wherein the H1 antihistamine is chlorcyclizine (CCZ), fexofenadine (FXF), or cetirizine (CET).

Embodiment 25. The method of any one of Embodiments 14-24, wherein the H1 antihistamine is chlorcyclizine (CCZ).

Embodiment 26. The method of any one of Embodiments 15-25, wherein the H2 antihistamine is cimetidine (CMT) or ranitidine (RNT).

Embodiment 27. The method of any one of Embodiments 15-26, wherein the H2 antihistamine is cimetidine (CMT).

Embodiment 28. The method of any one of Embodiments 13-27, further comprising identifying the subject as having protoporphyria related hepatobiliary disease prior to administering the antihistamines to the subject.

Embodiment 29. A method of treating protoporphyria related skin disease in a subject in need of, comprising administering an H1 antihistamine to the subject.

Embodiment 30. A method of treating protoporphyria related skin disease in a subject in need of, comprising administering an H2 antihistamine to the subject.

Embodiment 31. A method of treating protoporphyria related skin disease in a subject in need of, comprising administering an H1 and an H2 antihistamine to the subject.

The present invention will be described in greater detail by way of specific examples. The following examples are offered for illustrative purposes and are not intended to limit the invention in any manner. Those of skill in the art will readily recognize a variety of noncritical parameters which can be changed or modified to yield essentially the same results.

### EXAMPLES

### Example 1. The Histamine Pathway is a Target to Treat Hepatic Experimental Erythropoietic Protoporphyria

Erythropoietic protoporphyria (EPP) is caused by mutations in ferrochelatase which inserts iron into protoporphyrin-IX (PP-IX) to generate heme. EPP is characterized by PP-IX tissue accumulation, potentially leading to skin photosensitivity, cholestasis, and end-stage liver disease. Despite available drugs that may address photosensitivity to some extent, effective treatment of EPP-related liver disease remains an unmet need. In this Example, we administered delta-aminolaevulinic acid (ALA) and deferoxamine (DFO), which results in PP-IX overproduction and accumulation in model animal. High-throughput compound library screening of ALA+DFO-treated zebrafish was used to identify chlorcyclizine (first generation H1-antihistamine receptor blocker), as a drug that markedly reduces zebrafish liver PP-IX levels. The effect of chlorcyclizine was validated in porphyrin-loaded mouse hepatocytes, transgenic Fech^{m1Pas} EPP mice, and mice fed with the porphyrinogenic compound 3,5-diethoxycarbonyl-1,4-dihydrocollidine (DDC). Plasma and tissue PP-IX were measured by fluorescence; livers were analyzed by histology, immunoblotting, and qPCR. Chlorcyclizine-treated zebrafish larvae, DDC-fed and transgenic EPP mice manifested reduced hepatic PP-IX levels compared to controls. Histamine increases PP-IX accumulation in porphyrin-stressed hepatocytes, while H1/H2-receptor blockade decreases PP-IX levels. In both mouse models, chlorcyclizine lowers PP-IX level in female but not male mice in liver, circulating erythrocytes, and bone marrow, coupled with improved liver injury, decreased porphyrin-triggered protein aggregation and oxidation, and increased clearance of stool PP-IX. In mouse hepatocytes, chlorcyclizine induces nuclear translocation of constitutive androstane and farnesoid X receptors, and transactivates bile acid transporter expression. Chlorcyclizine also reduces hepatic mast cell number and histamine level in EPP mice. Histamine plays an important role in PP-IX accumulation in zebrafish and two experimental EPP models. Chlorcyclizine and/or other antihistamines, provide a potential therapeutic strategy to treat EPP liver disease via their function of decreasing PP-IX accumulation.

### Introduction

Erythropoietic protoporphyria (EPP, OMIM: 177000) is caused by the loss of ferrochelatase activity below 30% of normal, leading to accumulation of protoporphyrin-IX (PP-IX) in the liver and in bone marrow erythroid precursors¹⁻³. Skin photosensitivity is a major manifestation of EPP³, and 10-25% of EPP patients develop liver dysfunction and gallstones^{1,3} with 1-4% of overall cases progressing to end-stage liver failure needing transplantation.^{4,5} Despite availability of drugs that may relieve skin symptoms^{6,7}, a mechanism-based therapeutic for EPP-related liver disease remains an unmet need.

Using our previously described zebrafish EPP model⁸, we screened 2560 approved and bioactive compounds in the Prestwick Chemical Library® and the Library of Pharmaceutically Active Compounds (LOPAC®) and identified the H1-antihistamine chlorcyclizine (CCZ) as a potent PP-IX lowering agent. In mice, EPP can be modeled genetically by the homozygous Fech^{m1Pas} (fch/fch) mice⁹ or pharmacologically by feeding mice with a diet containing 3,5-diethoxycarbonyl-1,4-dihydrocollidine (DDC)¹⁰. In fch/fch and DDC-fed mice, CCZ exerted a dramatic female-selective effect of clearing PP-IX. CCZ led to nuclear translocation of the constitutive androstane and farnesoid X receptors from the cytoplasm, driving transcription of the multidrug resistance-associated protein-4 and bile salt export pump and increased PP-IX excretion. In primary mouse hepatocyte cultures, CCZ also inhibited the stimulatory effect of histamine on porphyrin synthesis and reduced PP-IX mediated protein aggregation.

The involvement of the histamine pathway in EPP led us to further investigate the potential role of mast cells whose increased presence may have been implicated in several liver diseases^{11,12}. In fch/fch mice, we observed elevated plasma and hepatic histamine levels with liver mast cell infiltrates. Notably, CCZ administration reduced hepatic mast cell number and histamine level. Taken together, histamine inhibition, as demonstrated by CCZ and other antihistamines, represents a unique therapeutic strategy to repurpose for treating EPP-associated liver disease.

### Materials and Methods

### Animals

All animal experiments were followed as approved by the Institutional Animal Care and Use Committee at Rutgers University and in accordance with the “Guide for the Care and Use of Laboratory Animals” published by the National Institutes of Health. Table-S1 lists the commercial chemicals and reagents used in the study.

### Chemical Libraries

The two compound libraries we used for our screening were the Prestwick Chemical Library® (Prestwick Chemical) and the Library of Pharmaceutically Active Compounds (LOPAC® 1280, Sigma Aldrich). Each of the two libraries comprises 1280 compounds that were purposely selected to represent all the major drug classes. In the Prestwick library, nearly 65% are approved by the FDA, and an additional 30% are approved by other agencies outside of the United States. For the LOPAC compounds, 25% are in the market, and the remaining 75% are bioactive molecules that have well-defined activities and a wide range of protein targets.

### Primary Drug Screen in a Zebrafish EPP Model

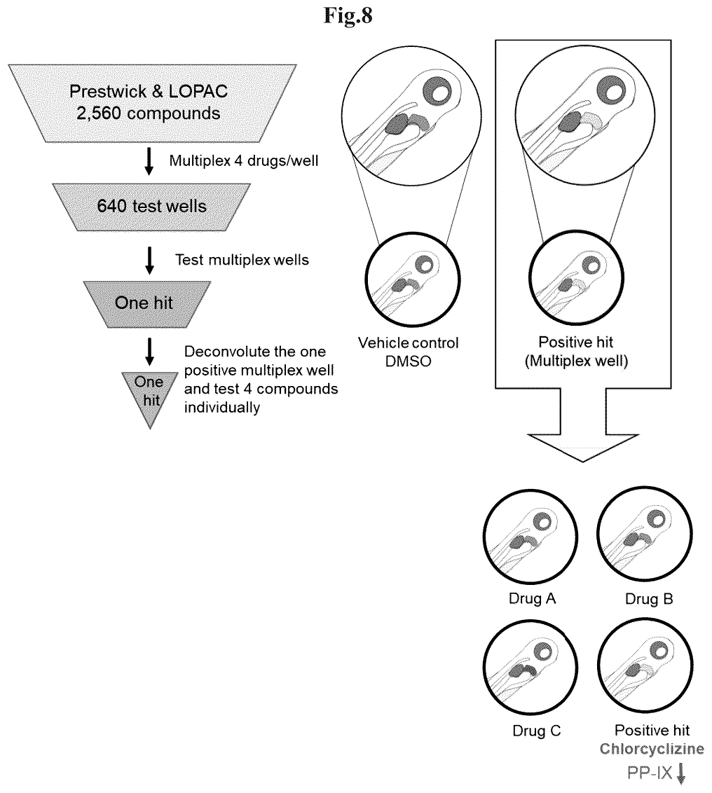

Unbiased high-throughput drug screening was performed using an fabp10-fgb-egfp transgenic line of ABTL zebrafish (A3 line) that expresses the liver-specific protein fibrinogen-β, tagged with green fluorescent protein as previously described¹³ and outlined in FIG. 8.

At 6-days post-fertilization (6 dpf), the A3 larvae were retro-orbitally injected with 2 nanoliters of δ-aminolevulinic acid hydrochloride (ALA, 50 mg/ml) and deferoxamine mesylate (DFO, 50 mg/ml). Control larvae were injected with water (the control vehicle). Injected larvae were then incubated in E3 fish medium containing four Prestwick or LOPAC library compounds or the drug vehicle, dimethyl sulfoxide (DMSO). Each of the eight 384-well plates from the two libraries were multiplexed (4 compounds/well, each tested at 2 and 5 μM) into 96-well plates to maximize resources and time efficiency.

After injection, larvae were incubated in the dark for 24 hours before they were anesthetized and imaged with an Image Xpress Micro-imager (Molecular Devices, San Jose, CA) using two fluorescence readouts, fluorescein isothiocyanate (FITC) and Texas Red. The FITC channel was used to trace the green-fluorescent livers. The traced livers were superimposed on the Texas Red channel where the porphyrin signal was quantified. A positive hit was determined as a reduction in the red fluorescence porphyrin signal in the liver (FIG. 8). Wells with a positive readout were subsequently deconvoluted to identify which of the four compounds in the positive wells accounted for the reduction. Chlorcyclizine, which consistently and markedly reduced the red porphyrin signals, was selected for subsequent confirmation assays.

### In Vitro EPP Model, Drug Treatment, and Porphyrin Measurement

Primary mouse hepatocytes were challenged with ALA+deferoxamine (DFO) (1 mM and 100 μM, respectively) that results in porphyrin accumulation, then treated with histamine, CCZ, or other histamine blockers. Cells were collected at indicated time points. Cell lysates and media porphyrins were prepared and measured as described¹⁴. In brief, the excitation wavelength was set at 400 nm, and the maxima PP-IX emission spectra was measured at 660 nm. A standard curve was generated using free acid PP-IX (Enzo). Cell lysates and culture media porphyrin levels were normalized to the protein content/sample, and then expressed as ratio of the fluorescence unit detected from the untreated ALA+DFO group.

### Primary Mouse Hepatocyte Isolation

Primary mouse hepatocytes were isolated from 8-10 weeks old female FVB/N mice as described¹⁵. In brief, mice were anesthetized with 40 mg/kg pentobarbital (Vortech Pharmaceuticals, Ltd.) before the livers were perfused with Hank's balanced salt solution containing 0.5 mM EGTA, 5.5 mM glucose, and 1% penicillin-streptomycin. Livers were then perfused with William's medium E (Gibco) and 40 U/L of collagenase IV (Worthington Biochemical Corporation). Liver parenchyma was then filtered through a 70-μM cell strainer (Fisher Scientific) and pelleted by centrifugation (50 g, 2 minutes, 4° C.). Cells were then seeded and allowed to attach for 6 h before treatment.

### In Vivo Efficacy Studies in Fech^{m1Pas} and DDC Mouse Model

5-6 weeks old Fech^{m1Pas} (fch/fch) mice were treated by intraperitoneal injection of CCZ (25 mg/kg) for 5 or 10 consecutive days. For the DDC model, 9-weeks old BALB/c mice were fed with a regular chow diet containing 0.1% DDC and co-administered with CCZ (25 mg/kg, i.p.) for 5 consecutive days. Liver, erythrocytes, bone marrow, plasma, bile, and stool were collected for subsequent porphyrin quantification and fluorescence measurement. A subset of mice was single-housed in wired bottom cages where stools were collected every 48 h. Parts of the livers were fixed with 10% formalin, stained with hematoxylin-eosin then subjected to quantification of porphyrin deposits using Image J software. May-Grunwald Giemsa staining was performed to assess mast cell presence. Details related to sample preparation and porphyrin fluorescence measurement for each tissue are described¹⁴.

Quantitative Real-Time PCR (qPCR)

Primary mouse hepatocytes were challenged with ALA+DFO and treated with DMSO or CCZ for 12 h then homogenized using QIAshredder (Qiagen). Total RNA was isolated (RNeasy mini kit, Qiagen) and purified according to manufacturer instructions. qPCR was performed with an iQ™ real-time PCR detection system (Bio-Rad) and SYBR green for monitoring cDNA amplification (Table-S2 lists the primers used). Samples were analyzed in duplicates, and five individual mice were tested for each group. 18s RNA was used as an internal control and transcript levels relative to 18s RNA were determined and reported as the mean±SD.

### Immunofluorescence Staining and Protein Analysis by Immunoblotting

Cells or livers were lysed in 2% SDS-containing sample buffer. Nuclear and S9 (which consists of microsomes and cytosol) fractions were prepared in 1% NP40 buffer (4° C.) and isolated following centrifugation at 500 g (4° C., 10 min). Protein oxidative status was analyzed globally using the Protein Carbonyl Assay Kit (Abcam) as recommended by the manufacturer. For staining, liver sections were fixed in ice-cold methanol (20 min) then processed using standard procedures. Table-S3 lists the antibodies used in the study.

### Statistics

Statistical significance was assessed with GraphPad Prism 8.0 software. Data are presented as means±SD (n≥3). Student's t-test or one-way ANOVA was used to determine any statistically significant differences between the means of two; or three or more independent groups. Error bars represent standard deviation of the mean (*p<0.05/**p<0.01/***p<0.001/****p<0.0001).

### Results

### Quantitative High-Throughput Drug Screening LED to the Identification of CCZ as a Porphyrin-Lowering Agent

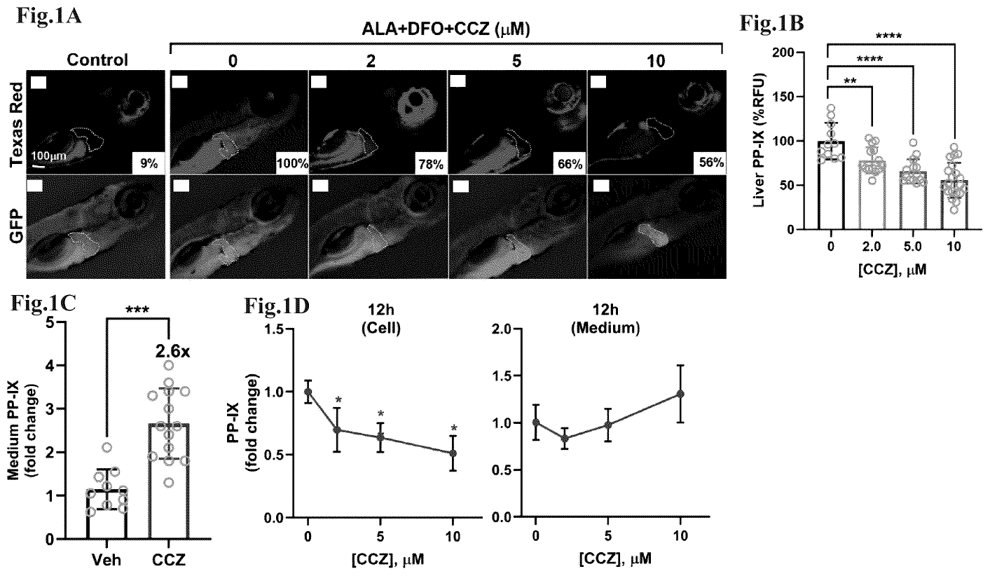

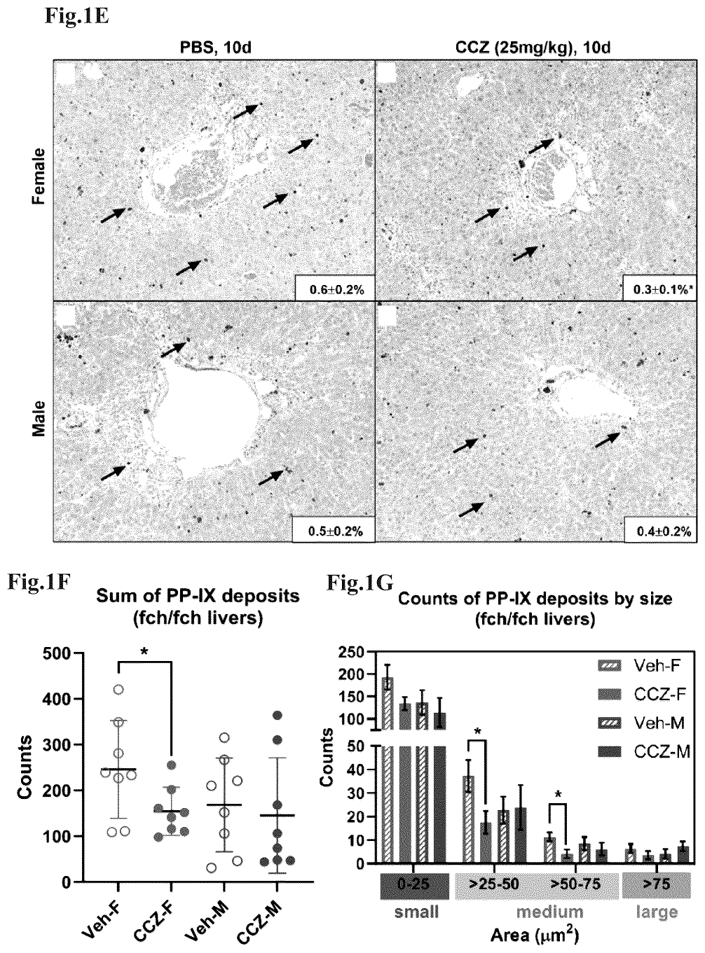

An unbiased, quantitative high-throughput drug screening (qHTS) of the Prestwick Chemical Library® and the Library of Pharmaceutically Active Compounds® was carried out using a precursor-induced zebrafish EPP model that we previously established⁸ (FIG. 8). In this model, overproduction of endogenous PP-IX was induced by retro-orbital injection of the heme precursor δ-aminolevulinic acid hydrochloride (ALA) and the iron chelator deferoxamine mesylate (DFO) into zebrafish A3 larvae. When compared to water-injected control (FIG. 1A, the first upper panel (Control)), co-administration of ALA and DFO resulted in elevated PP-IX synthesis and subsequent accumulation in the liver and biliary system (FIG. 1A, the first upper panel (Control), and the second upper panel (0 μM CCZ)). Of the 2560 compounds that were screened, the H1-antihistamine chlorcyclizine (CCZ) was selected for further study based on consistent robust reduction in hepatic PP-IX levels. When evaluated at higher doses, CCZ led to greater decrease in hepatic PP-IX accumulation (FIGS. 1A,1B), and a 2.6-fold increase in excreted PP-IX level in the fish growth medium (FIG. 1C). We then validated the initial drug screen result in cultured mouse hepatocytes treated with ALA+DFO and measured PP-IX fluorescence in the cell lysates and medium (FIG. 1D). In support of the primary screen result in zebrafish, CCZ similarly decreased intracellular PP-IX level in a dose dependent manner. Per our cell-based assay, there was no significant change in medium PP-IX level following CCZ treatment.

### CCZ Induces PP-IX Clearance and Reduces Liver Injury in Female Fch/Fch Mice

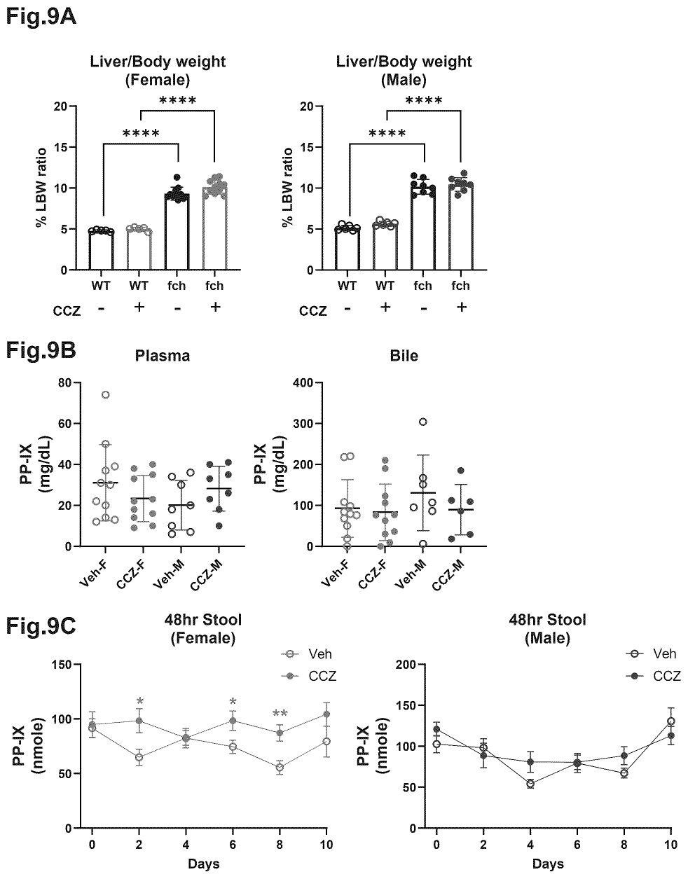

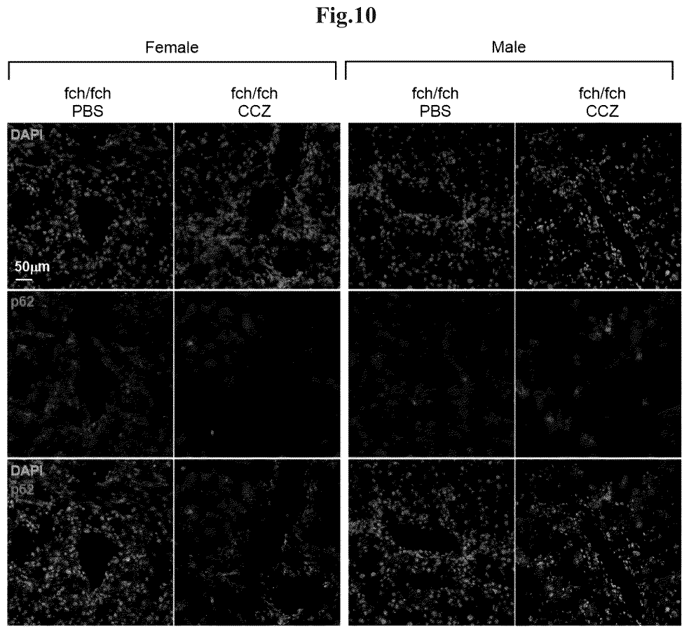

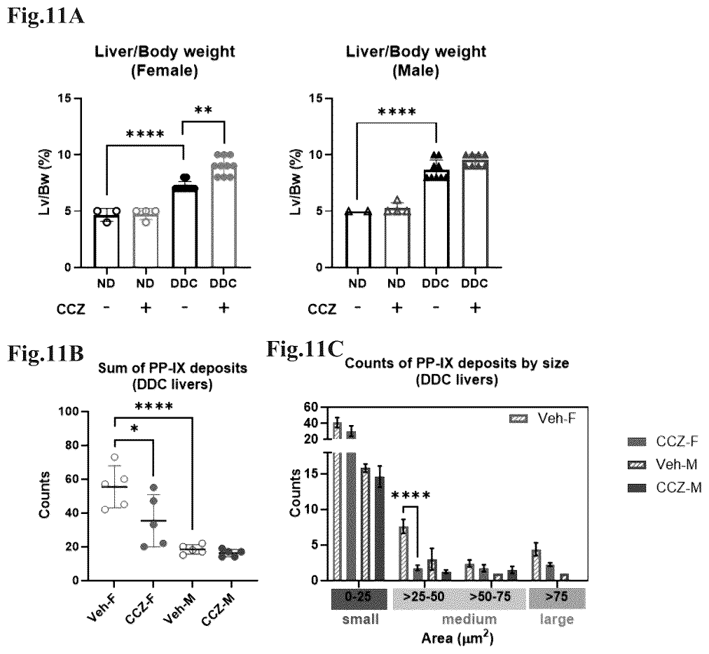

Fech^{m1Pas} (fch/fch) mice were previously reported to closely phenocopy the more severe forms of human erythropoietic protoporphyria (EPP)⁹. As expected, fch/fch mice display hepatomegaly with a liver-to-body weight ratio nearly double the ratio in wild-type mice (FIG. 9A). In the fch/fch livers, particulate PP-IX deposits were found to accumulate mostly in the bile canaliculi and interlobular ducts (FIG. 1E, left upper panel and left lower panel). The extent of hepatic PP-IX accumulation was assessed by the percent area (FIG. 1E) and total counts (FIG. 1F) of the dark brown PP-IX deposits (details related to quantification of the PP-IX area and counts are described in the Supplementary Data “ImageJ Workflow for Liver PP-IX quantification”). Consistent with what we observed in zebrafish and cultured mouse hepatocytes, administration of CCZ (25 mg/kg) led to significant reduction in the hepatic PP-IX area and counts in female, but not male, mice. To further analyze the profile of the hepatic PP-IX deposits, we sorted each PP-IX deposit into three different size categories: small (0-25 μm²), medium (>25-75 μm²), and large (>75 μm²). Our analysis revealed that the overall observed reduction of deposits in female was entirely accounted for by medium-sized PP-IX deposits (FIG. 1G).

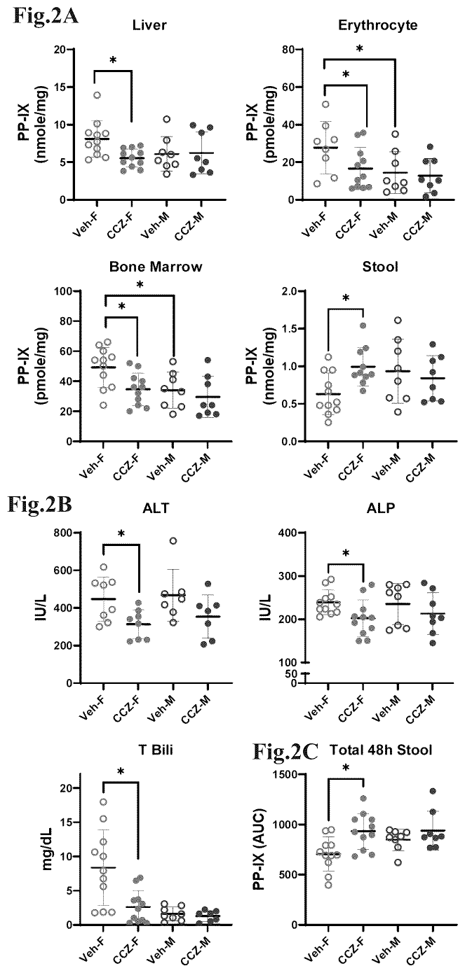

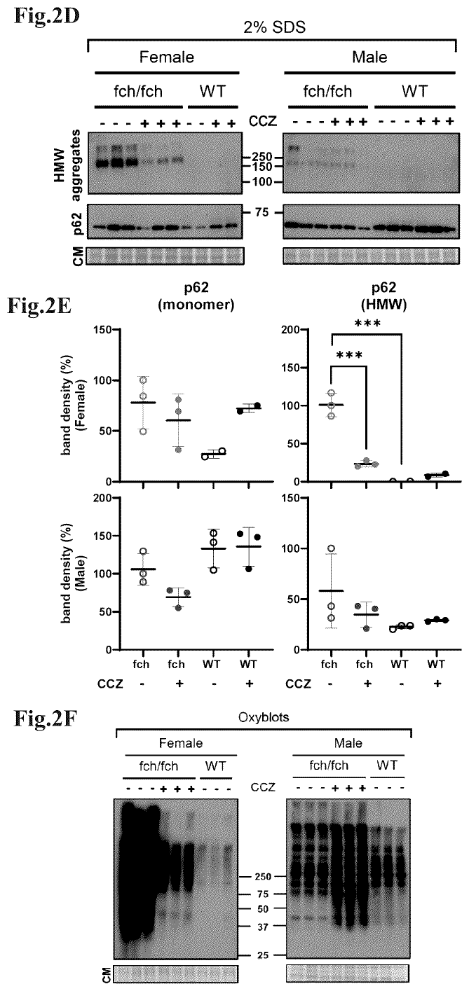

To determine whether CCZ changes porphyrin levels in tissues aside from the liver, we measured PP-IX in known target tissues such as the liver, erythrocytes, bone marrow, and plasma, as well as key routes that PP-IX takes to exit the body, which are the bile and stool^{9,16}. Notably, in female but not male mice, CCZ reduced PP-IX levels in the liver, erythrocytes, and bone marrow, while increasing PP-IX excretion into stool (FIG. 2A). There was no significant change in plasma and bile PP-IX levels (FIG. 9B). CCZ treatment also led to decreased plasma liver injury markers alanine aminotransferase (ALT), alkaline phosphatase (ALP), and total bilirubin (FIG. 2B). Total stool PP-IX output during the entire 10-day study period increased in CCZ-treated females when compared to their vehicle-treated control (FIG. 2C, FIG. 9C). Taken together, our data suggest CCZ treatment for as little as 10 days is sufficient to drive clearance of PP-IX from several target tissues into the stool.

### CCZ Reduces Porphyrin-Mediated Protein Aggregation in Female Fch/Fch Liver

Accumulation of PP-IX leads to organelle-selective protein aggregation, thereby causing hepatocellular damage^{14,17}. One previously identified protein prone to prominent aggregation upon porphyrinogenic stress is p62/SQSTM1¹⁷, an adaptor protein involved in the ubiquitin-proteasome machinery. In fch/fch total liver extracts, p62 forms high molecular weight (HMW) aggregates that are resistant to dissociation in 2% SDS-containing buffer (FIG. 2D). CCZ treatment reduced p62 aggregation in female and not male livers (FIGS. 2D,2E). When assessed by immunofluorescence staining, the p62 signal was mostly diffuse, except a handful of puncta that were scattered throughout the liver section (FIG. 10). Consistent with the immunoblot findings, the overall p62 signal decreased dramatically in the CCZ-treated female but not male livers (FIG. 10).

To globally assess the status of protein oxidation, we performed a protein carbonyl assessment of wild-type and fch/fch liver lysates. The oxyblots revealed that compared to wild-type livers, fch/fch livers bore a much greater oxidative burden, likely due to the chronic porphyrinogenic stress (FIG. 2F). While CCZ markedly reduced overall protein oxidation in female livers, there was no significant effect in male livers (FIG. 2F).

### CCZ Reduces PP-IX Level and Injury in Female DDC Liver

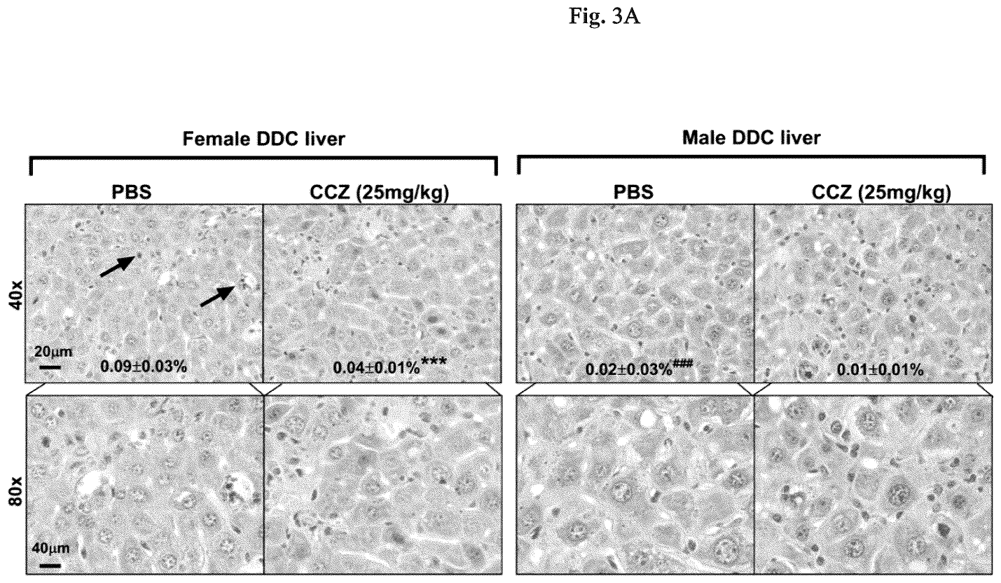

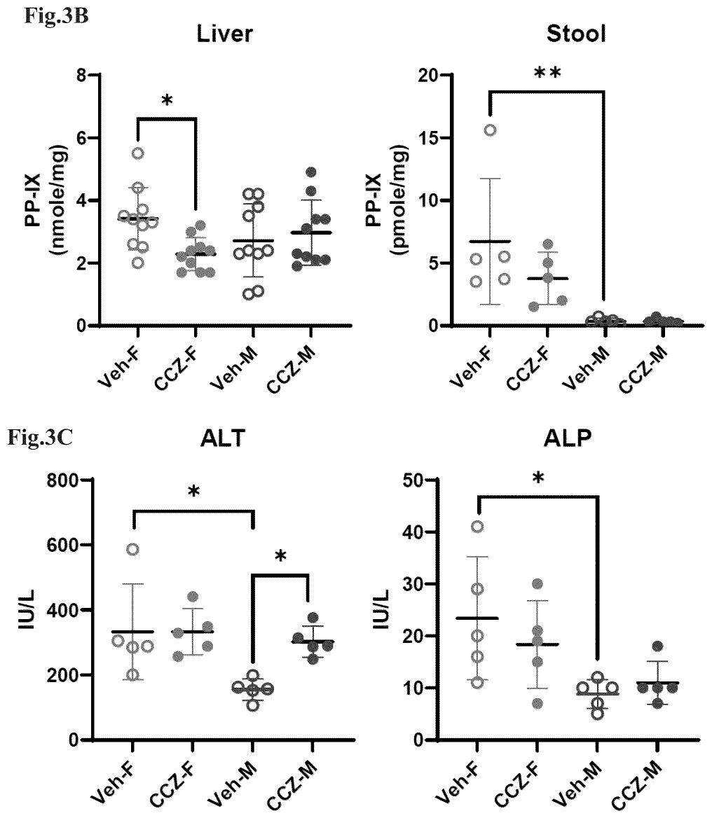

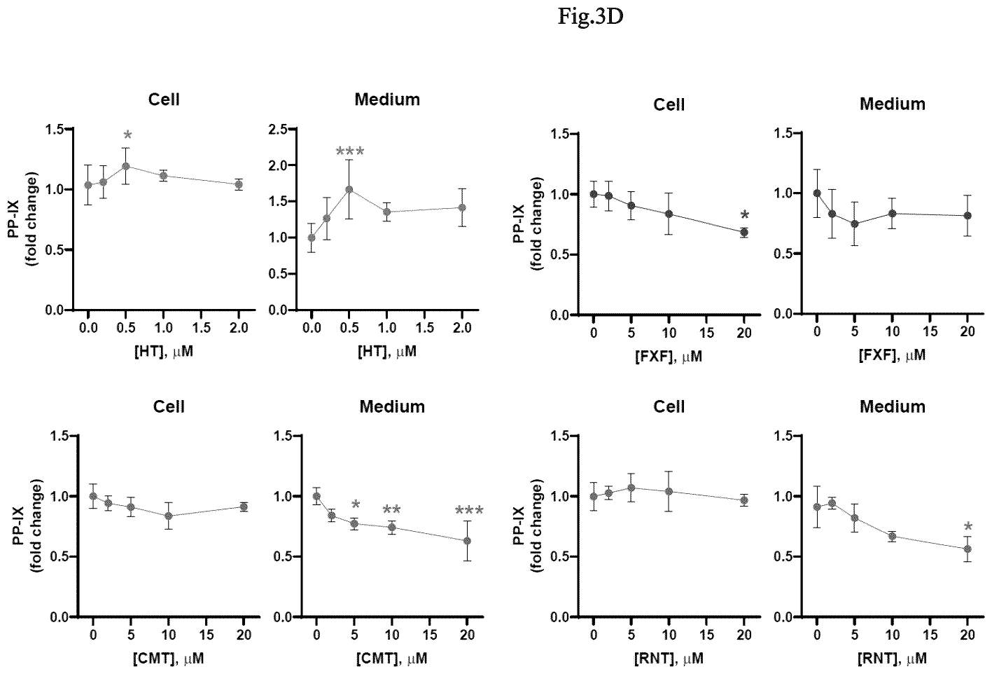

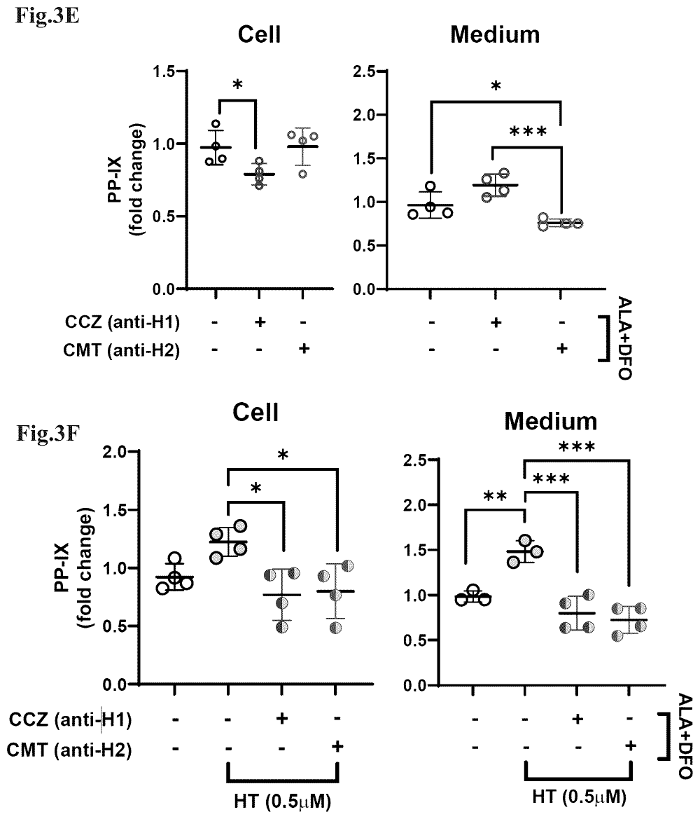

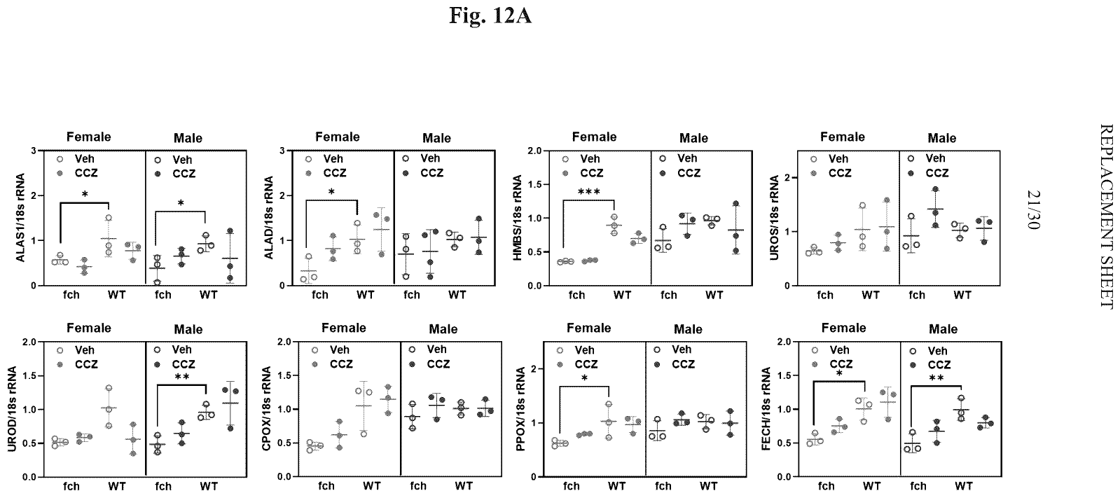

Some aspects of EPP can also be modeled by feeding mice with 3,5-diethoxycarbonyl-1,4-dihydrocollidine (DDC), whose metabolite N-methyl PP-IX inhibits ferrochelatase^{10,17}. Females were known to generate more PP-IX than males because they express higher levels of CYP3A1, which converts DDC into N-methyl PP-IX^{10,17}. Similarly, in our case, after 5 d of DDC feeding, all mice had enlarged livers (FIG. 11A), but a greater hepatic PP-IX area and counts, as well as excreted PP-IX in stool, was seen in females than males (FIGS. 3A,3B). CCZ treatment led to significant reduction in PP-IX level in the female but not male livers (FIG. 3B). Similar to female fch/fch liver, the PP-IX deposits most reduced by CCZ were within the medium-size >25-50 μm² range (FIGS. 11B,11C). CCZ treatment did not cause significant changes in plasma ALT, ALP, or total bilirubin levels in DDC-fed female mice but increased plasma ALT and total bilirubin levels in DDC-fed male mice (FIG. 3C), possibly due to hepatoxic drug-drug interaction between DDC, CCZ, and their metabolites.

### Histamine is Porphyrinogenic and can be Blocked by H1 and H2 Antihistamines

To determine whether CCZ acts through the histamine receptor signaling pathway, we performed a dose response study for histamine and its blockers using primary mouse hepatocytes challenged with ALA and DFO. Of the four histamine receptor subtypes, H1 and H2 are expressed in the liver across multiple species including zebrafish^{18,19}, mouse^{11,19}, and human¹⁹⁻²¹. At 0.5 μM, histamine modestly increased PP-IX synthesis in hepatocytes by nearly 20% but greatly increased medium PP-IX by nearly 70% (FIG. 3D). This stimulatory effect disappeared when it was given at higher doses, possibly due to cytotoxicity.

To complement our findings with CCZ, which is a first-generation H1-blocker, we tested fexofenadine, a second-generation H1-blocker, and the two acid-suppressor H2-blockers cimetidine and ranitidine. Cimetidine (CMT) is of particular interest because it has previously been described to inhibit ALA synthase-1²² and is currently undergoing a phase-2 clinical trial for the treatment of human EPP (ClinicalTrial.gov: NCT05020184)²³. In our in vitro EPP model, fexofenadine reduced cell PP-IX level, and the H2 blockers CMT and ranitidine lowered medium PP-IX levels but did not affect cell PP-IX levels (FIG. 3D).

We then tested CCZ and CMT side-by-side in the presence and absence of histamine. As expected, CCZ reduced intracellular PP-IX by 20%, while CMT did not have a significant effect; conversely, CMT reduced medium PP-IX level by 24%, and CCZ did not show a significant effect (FIG. 3E). However, when histamine was present, which increases intracellular PP-IX by 20%, both CCZ and CMT reverse the histamine effect and further decrease cell PP-IX by an additional 20% (FIG. 3F). Similarly, medium PP-IX levels increased 50% by histamine, and this increase was inhibited by CCZ and CMT (FIG. 3F). In summary, given that H1- and H2-blockade led to PP-IX reduction in different compartments (cell vs medium), it is likely that H1- and H2-antihistamines act on different signaling pathways.

### CCZ Decreases Mast Cell Presence and Histamine Level in the Fch/Fch Liver and Plasma

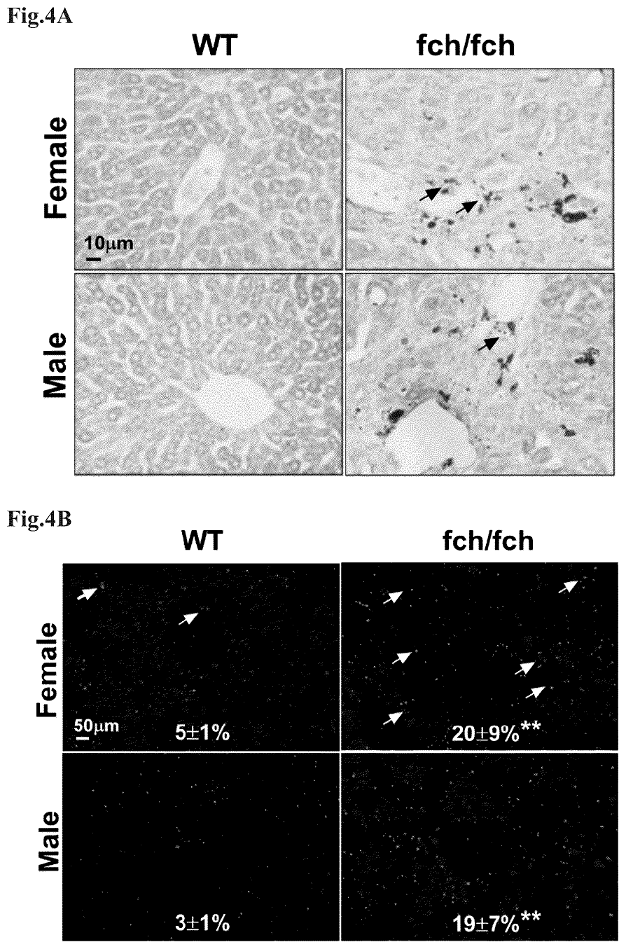

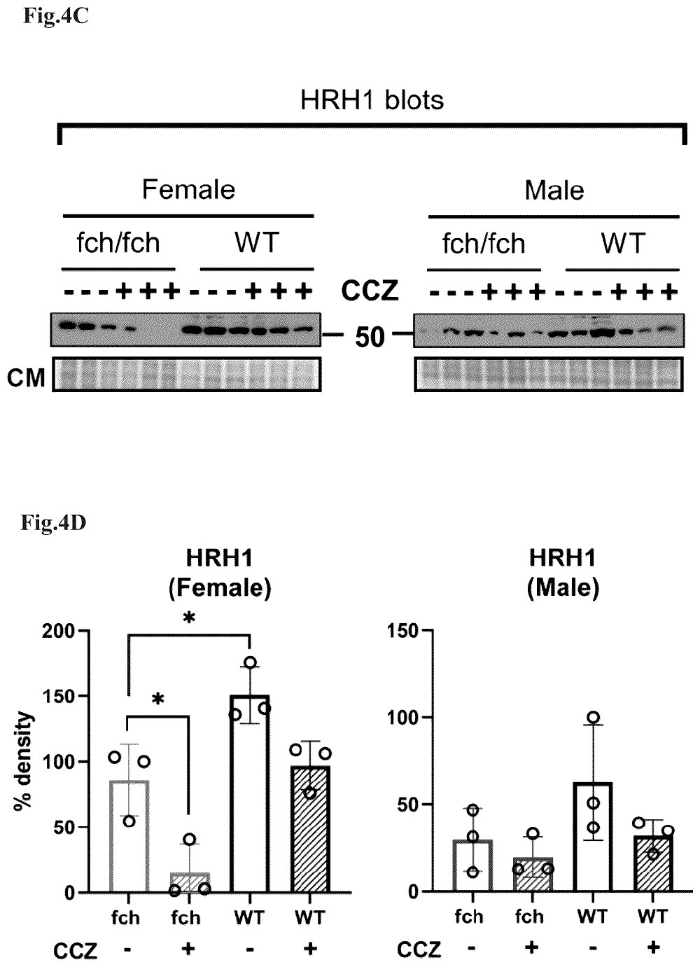

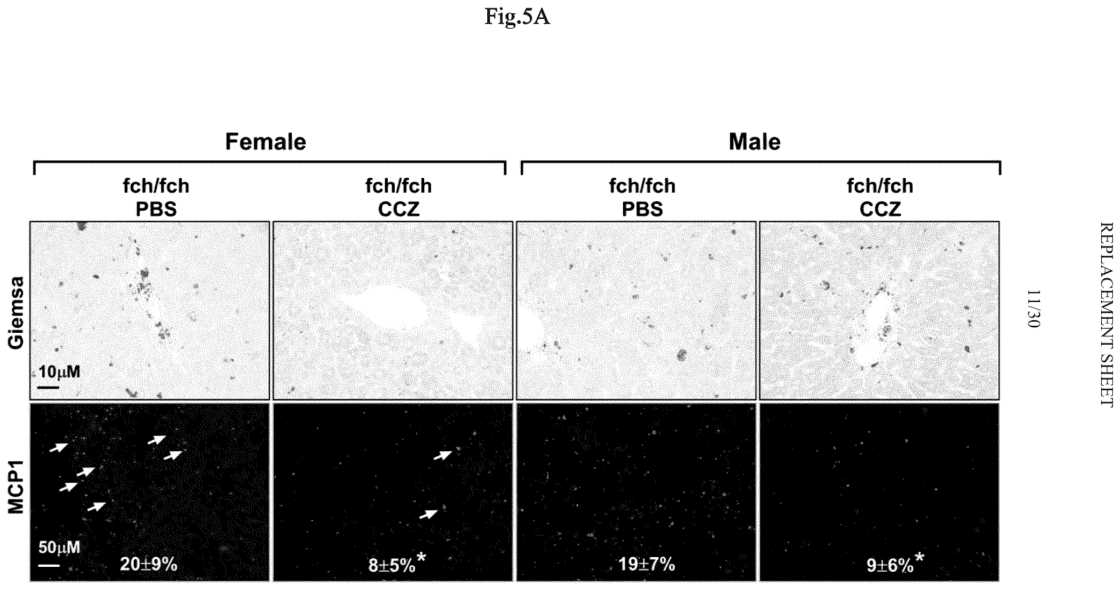

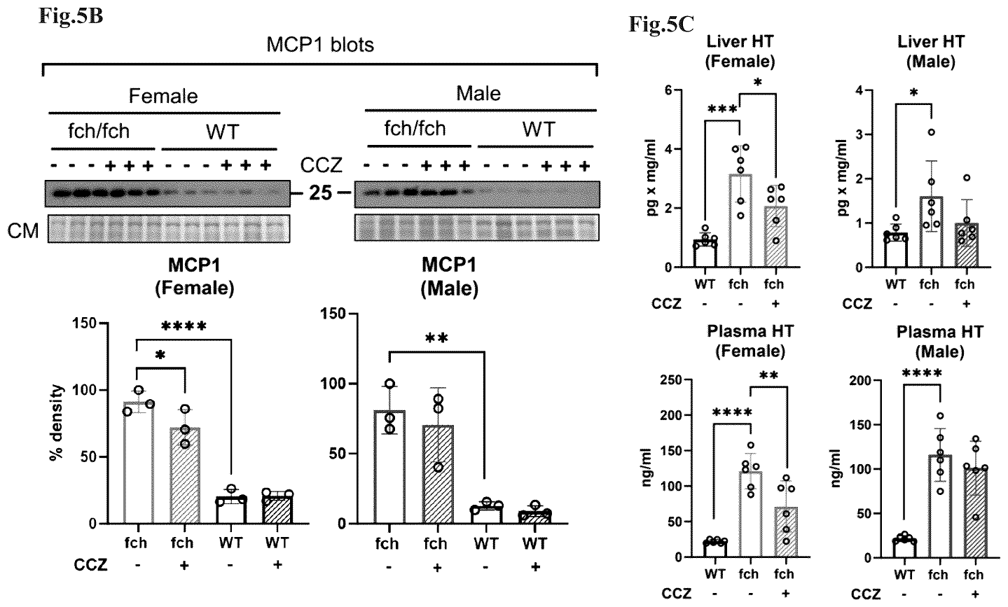

Since mast cell (MC) is a major source of histamine in the body, we evaluated their abundance in the liver using Giemsa staining and immunofluorescence staining of mast cell protease-1 (MCP1). Compared to wild-type livers, which show minimal Giemsa or MCP1 staining, both male and female fch/fch livers display heightened MC presence (FIGS. 4A,4B). Baseline histamine receptor type 1 (HRH1) expression is reduced in female fch/fch livers when compared to wild-type livers, with CCZ administration leading to a further decrease in HRH1 expression in female livers (FIGS. 4C,4D). Notably, CCZ treatment of fch/fch mice depleted MC in livers of both sexes (FIG. 5A), but protein levels of MCP1 as determined by immunoblotting decreased more prominently in female livers (FIG. 5B). Importantly, hepatic and plasma histamine levels increase dramatically in fch/fch mice (compared with wild-type controls), and this increase was reversed by CCZ only in the female mice (FIG. 5C).

### CCZ Transactivates Hepatic CAR and FXR Receptors in a Female-Selective Manner

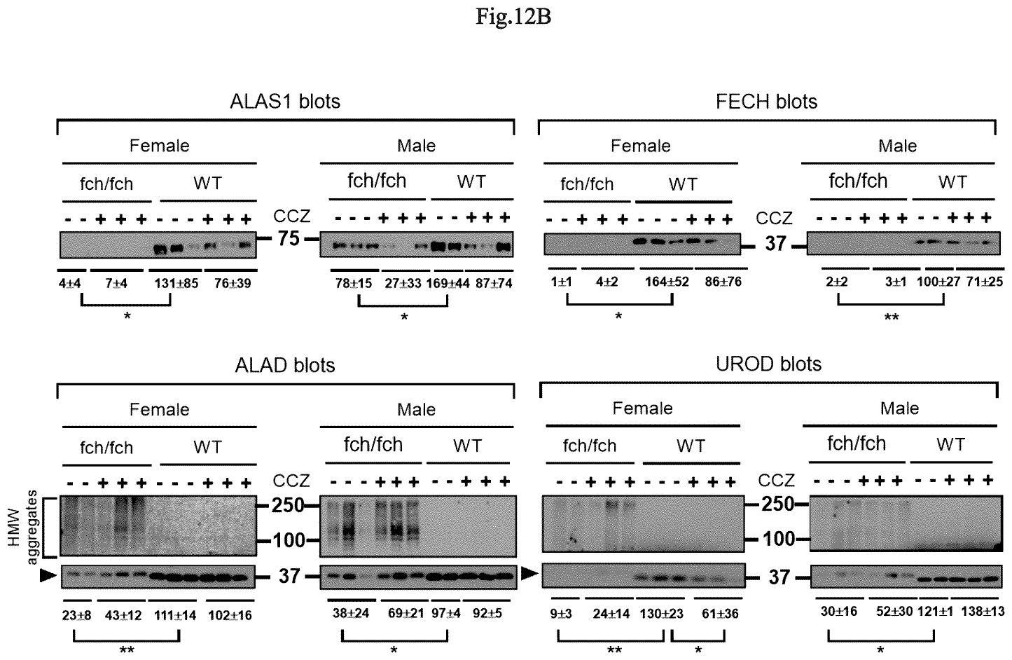

Similar to what was reported in human EPP, in terms of a compensatory response to excess PP-IX production²⁴, mRNA expressions of all eight heme biosynthetic enzymes in fch/fch mice of both sexes were either significantly suppressed or lower than the mean expression detected in wild-type mice (FIG. 12A). This loss of expression was also reflected at the protein level (FIG. 12B). CCZ did not alter the mRNA or protein expression of these enzymes, thereby suggesting that CCZ likely does not act on PP-IX biosynthesis.

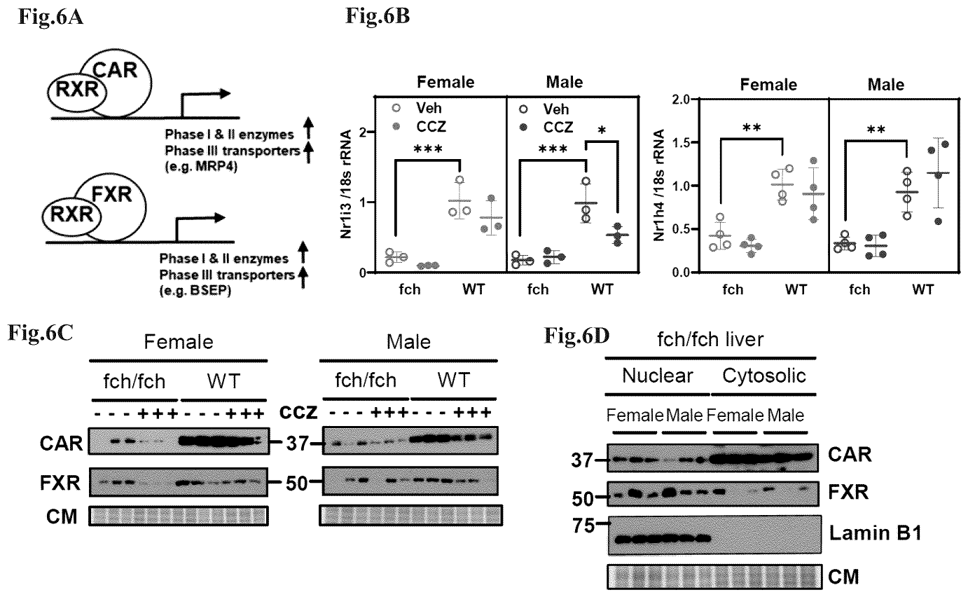

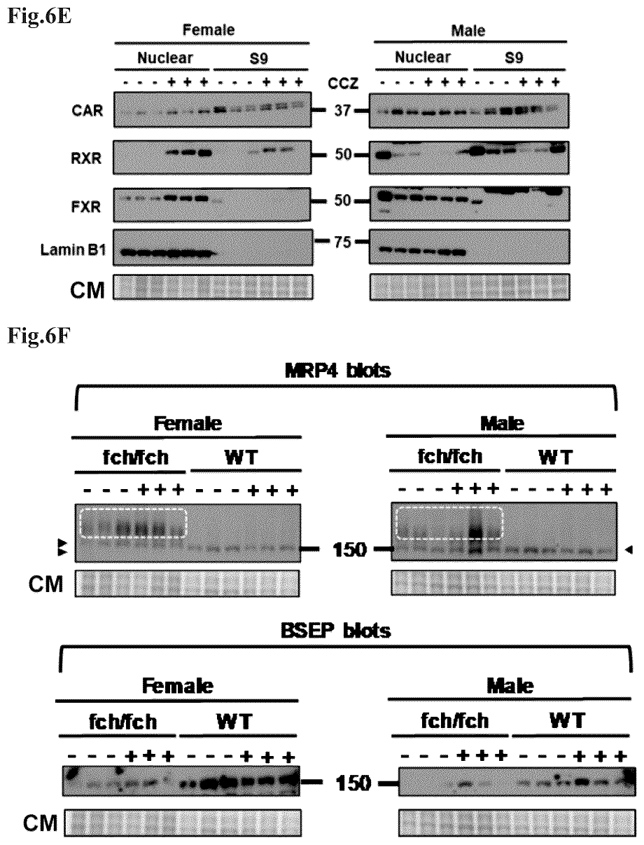

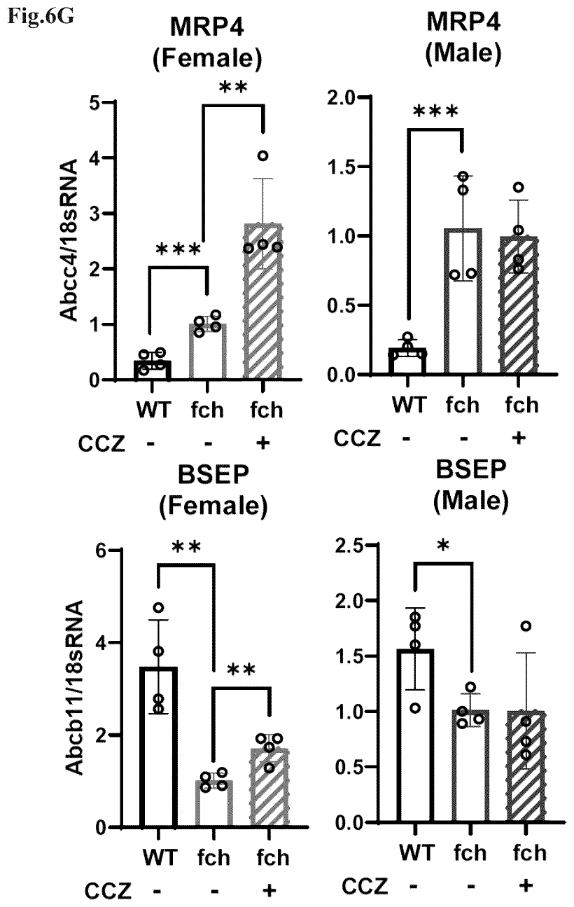

To further pursue the possibility that CCZ enhance hepatic PP-IX clearance via excretion, we explored several key regulators in the liver that drive bile acid detoxification and export. Constitutively active androstane receptor (CAR), the bile acid receptor farnesoid-X receptor (FXR), and their common transactivation partner retinoid X receptor (RXR) act in this capacity^{25,26} (FIG. 6A). Both CAR and FXR mRNA and protein were significantly suppressed in fch/fch livers, and CCZ further decreased their protein expressions in female fch/fch livers (FIGS. 6B,6C). Under basal conditions, CAR and FXR reside in the cytoplasm, and upon activation both receptors enter the nucleus where they upregulate phase-I and phase-II detoxification enzymes and phase-III transporters^{26,27}. In fch/fch livers, a fraction of CAR and almost all FXR already translocate into the nuclear fraction (FIG. 6D). Notably, CCZ treatment further enriches nuclear presence of CAR, FXR, and RXR, but this increased nuclear partitioning is observed only in female, and not male, mice (FIG. 6E).

To verify that nuclear CAR and FXR induce elimination of PP-IX in bile by upregulating transporters, we examined the multidrug resistance-associated protein 4 (MRP4) and the bile salt export pump (BSEP), which are known respective targets of CAR^{28,29} and FXR^{25,30}. Hepatic MRP4 level is low but increases upon cholestasis, presumably as a defense mechanism to protect the liver from accumulation of cytotoxic bile acid²⁹. In fch/fch livers, MRP4 was induced both at the mRNA and protein levels (FIGS. 6F,6G). Although MRP4 protein was similarly upregulated in both sexes, MRP4 mRNA was further increased only in the female by CCZ. In the case of BSEP, both mRNA and protein were significantly suppressed in fch/fch livers, but CCZ reversibly upregulated its expression in females (FIGS. 6F,6G).

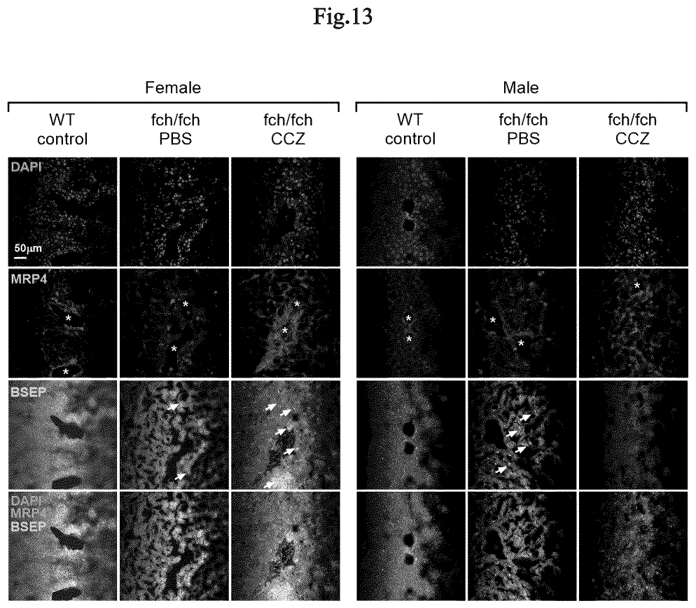

Immunofluorescence staining of MRP4 and BSEP also revealed a female-selective increase in their staining after CCZ treatment (FIG. 13).

### Discussion

Of the major therapeutics that had previously been attempted in the last four decades to treat EPP, only afamelanotide (SCENESSE®), an α-melanocyte-stimulating hormone analogue, received approval by the U.S. Food and Drug Administration in 2018³¹. This analogue was designed to target cutaneous EPP symptoms without addressing PP-IX accumulation in other organs. We performed a high-throughput drug screen using a porphyrin precursor administration zebrafish EPP model⁸ and identified the H1-antihistamine chlorcyclizine as a highly effective PP-IX-lowering agent in zebrafish liver. This led us to examine and show that histamine promotes porphyrin accumulation and that H1 and H2-receptor blockade could be a viable strategy for treating EPP-related liver disease. Whether histamine receptor blockade also plays a role in skin manifestations remains to be determined.

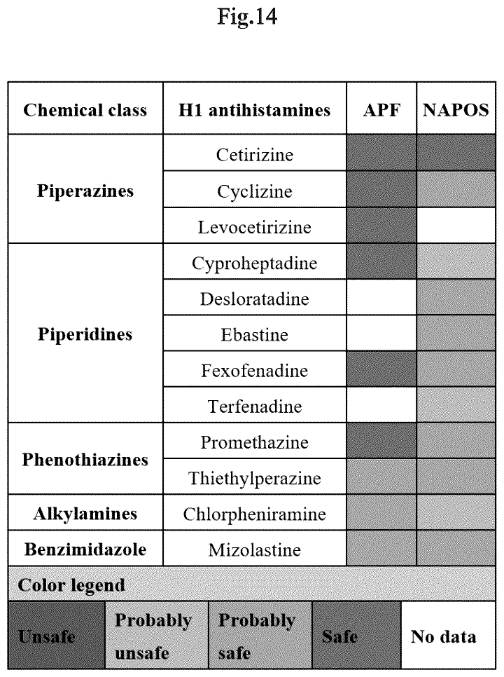

Chlorcyclizine (CCZ) and cimetidine are H1- and H2-antihistamines used to treat allergy, and gastroduodenal ulcers, respectively³². They act as inverse agonists by binding to H1- or H2-histamine receptors and inhibit binding of histamine and subsequent Gαq/11 and Gαs signaling^{21,32}. Upon surveying their safety profile, almost all H1/H2 antihistamines, with a few exceptions, are considered safe drugs for patients with porphyria based on recommendations provided by the American Porphyria Foundation (porphyriadrugs.com) and the Norwegian Porphyria Centre (www.drugs-porphyria.org/index.php) databases (FIG. 14). Although there is no reported safety data for CCZ in patients with porphyria, this first-generation antihistamine was approved in the 1940s and was reported to be a well-tolerated adjuvant antiviral agent for hepatitis C infection in human patients³³.

PP-IX deposition has been noted in the severe cases of human EPP liver disease^{34,35}. Here, we showed that histamine itself worsens PP-IX accumulation and release from isolated mouse EPP hepatocytes (FIGS. 3D,3F). The fch/fch mice also had elevated serum histamine levels and heightened mast cell presence in the liver (FIG. 4). These findings suggest that histamine likely plays a pathologic role in driving PP-IX build-up and subsequent disease manifestation in the liver. Limited studies have reported that the oral H1 antihistamine terfenadine or H2 antihistamine cimetidine helped reduce skin photosensitivity in EPP patients³⁶⁻³⁸ though the effect of histamine blockers on EPP-associated liver injury is unknown prior to the study herein. Protein aggregation and oxidation are key drivers to porphyrin-mediated hepatocellular and liver tissue injury^{8,14,17,39,40}. Our findings show that CCZ reduced protein aggregation and oxidation (FIGS. 2D-2F) and ameliorated cellular and liver tissue injury (FIGS. 1-3). CCZ also decreased HIHR expression (FIG. 4C), and together with other antihistamines showed a PP-IX lowering effect (FIG. 1D). Therefore, antihistamines offer an attractive drug class to be examined in human EPP-related liver disease.

Enhanced excretion of toxic bile, and presumably the PP-IX admixed with it, has been a strategy explored in EPP patients using bile acid sequestrants, cholestyramine, colestipol, and ursodeoxycholic acid⁴¹⁻⁴³. Although reports of their clinical benefits were anecdotal, they provided proof-of-concept that enhanced bile excretion can ameliorate both cutaneous and hepatic EPP complications. Our findings showed that CCZ led to coordinated induction of the CAR/MRP4 and FXR/BSEP transactivation systems (FIG. 6), and enhanced PP-IX excretion into the stool (FIG. 3). In support of our findings, MRP family members 5/9 have been described as heme exporters⁴⁴⁻⁴⁶, and a deleterious mutation in MRP9/ABCC12 causes cholestasis in zebrafish, mice and humans⁴⁷. Although the connection between the MRP family members, heme/porphyrin export, and cholestasis remains fragmentary, our data suggests that MRP4 may be a potential molecular target to enhance PP-IX clearance.

In both porphyria models we tested (fch/fch and DDC-fed mice), only females responded to CCZ treatment (FIGS. 2,3). This mouse female-selective phenotype was also noted at the molecular level with CCZ enriching nuclear CAR and FXR accumulation in female but not male fch/fch livers. Sex-selective effects in CCZ metabolism have not been described in mice or humans, though it has been described in rats that CCZ is metabolized eight times more efficiently in male versus female livers⁴⁸. Since we only observed protection in female fch/fch and DDC-fed mice, coupled with the reported male>female CCZ metabolism profile in rats, suggests that it is the parent compound CCZ, and not its demethylated metabolite, nor-chlorcyclizine, that has the active porphyrin-lowering effect in female mice. We are not aware of similar metabolism differences in humans.

CCZ is reported to have potential drug-drug interactions, likely due to its effect on some cytochrome P-450 enzymes⁴⁹. This provides a likely explanation for the observed ALT elevation in male but not female DDC-fed mice treated with CCZ (FIG. 3C). Notably, the intraperitoneal dose of CCZ that we used in mice (25 mg/kg) converts to a human dose of 2 mg/kg⁵⁰ which is likely compatible with safe dosing in humans of 50 mg oral tablet 2-4 times per day⁵¹.

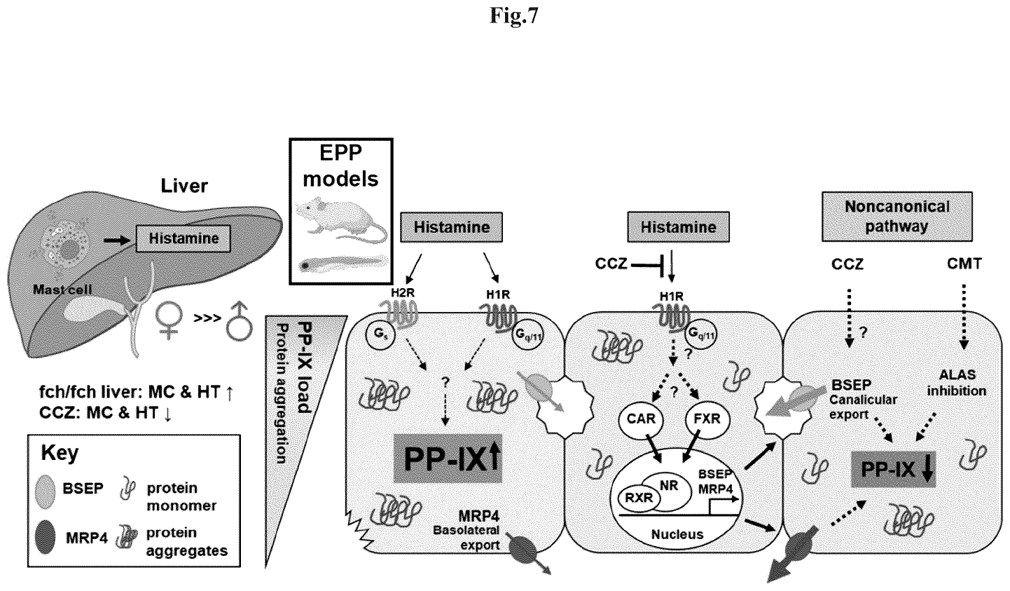

Two conceptual strategies to limit accumulation of toxic PP-IX involve inhibiting heme/porphyrin synthesis and/or clearing PP-IX through bile excretion. Our overall findings (FIG. 7) present compelling evidence that the H1-antihistamine chlorcyclizine adopted both mechanisms whereby (i) in the liver, it led to concerted induction of phase III transporters followed by increased bile/PP-IX excretion in zebrafish, isolated hepatocytes, and mouse models of EPP, and (ii) systemically, chlorcyclizine decreases circulating and liver histamine levels, which in turn suppresses PP-IX biosynthesis. Chlorcyclizine and cimetidine appear to have differing effects that may relate to their noncanonical effects which may also differ for specific H1 or H2 blockers. Although cimetidine has been described as an ALA synthase inhibitor²², recent findings suggest that this may not be the case⁵² though this may depend on the biologic context. Cimetidine is undergoing clinical trial to study its effect on photosensitivity in EPP²³. It remains to be investigated whether chlorcyclizine has an effect on skin manifestations of EPP, and whether its effect on bone marrow (FIG. 2A) extends to certain other antihistamines. Through its dual ability to clear PP-IX and reduce protein aggregation and oxidation, CCZ or potentially other H1-blockers represent an attractive and likely safe drug to be tested/used alone or in combination with cimetidine (or other H2-blockers) for EPP or XLP patients who are at risk for developing hepatobiliary complications.

Abbreviations: ALA, δ-aminolevulinic acid; ALT, alanine aminotransferase; ALP, alkaline phosphatase; BSEP, bile salt export pump; CCZ, chlorcyclizine; CMT, cimetidine; DDC, 3,5-diethoxycarbonyl-1,4-dihydrocollidine; DFO, deferoxamine mesylate; EPP, erythropoietic protoporphyria; fch/fch, Fech^{m1Pas}, H1, H2, histamine receptor types 1 and 2; HMW, high molecular weight; MC, mast cells; MRP4, multidrug resistance-associated protein 4; PP-IX, protoporphyrin IX; qHTS, quantitative high-throughput drug screening.

TABLE S1
Commercial chemicals and reagents used in the study.

| Reagents used in cell culture | Vendor | Catalog number |
|---|---|---|
| Protoporphyrin-IX | Enzo | ALX-430-041-G001 |
| δ-aminolevulinic acid | Sigma Aldrich | A7793-1G |
| hydrochloride | | |
| Deferoxamine mesylate salt | Sigma Aldrich | D9533-1G |
| Histamine ELISA kit | Abcam | ab213975 |

TABLE S2
The RT-qPCR primers used in the study.

| Transcript | Human (Homo sapiens) PCR primers | Mouse (Mus musculus) PCR primers |
|---|---|---|
| Alas1 | ID: Hs.PT.56a.40544386.g | ID: Mm.PT.58.7841429 |
| | 5′-ACATCACACAGCTCTTCCAG-3′ | 5′-TGACTACCTAGGCATGAGTCG-3′ |
| | (SEQ ID NO: 1) | (SEQ ID NO: 3) |
| | 5′-AAGTACATCTTCCGCCACAA-3′ | 5′-CTTGCTCGTTCCAGAAATATTCC-3′ |
| | (SEQ ID NO: 2) | (SEQ ID NO: 4) |
| Alad | ID: Hs.PT.56a.3971613 | ID: Mm.PT.58.29257918 |
| | 5′-AAGATCAAGACACAGCGTAGG-3′ | 5′-CCTCATCTATCCCATCTTTGTCA-3′ |
| | (SEQ ID NO: 5) | (SEQ ID NO: 7) |
| | 5′-AACCTCATCTACCCCATCTTTG-3′ | 5′-GGTCTCAGCATCTCTTCTAGC-3′ |
| | (SEQ ID NO: 6) | (SEQ ID NO: 8) |
| Hmbs | ID: Hs.PT.39a.22214823 | ID: Mm.PT.39A.22214827 |
| | 5′-AGGGTACGAGGCTTTCAATG-3′ | 5′-AAAGATGAGGGTGATTCGAGTG-3′ |
| | (SEQ ID NO: 9) | (SEQ ID NO: 11) |
| | 5′-CATGTCTGGTAACGGCAATG-3′ | 5′-AAGAATCTTGTCTCCCGTGG-3′ |
| | (SEQ ID NO: 10) | (SEQ ID NO: 12) |
| Uros | ID: Hs.PT.58.38508932 | ID: Mm.PT.58.13264122 |
| | 5′-TGTTCAGGTTCCCTTGGATTC-3′ | 5′-TGCCACCGCTTCTCTAGT-3′ |
| | (SEQ ID NO: 13) | (SEQ ID NO: 15) |
| | 5′CCTGTGGAAACCTCAAAAGAGA-3′ | 5′-TCTCCTTTGATAGTTCCACACG-3′ |
| | (SEQ ID NO: 14) | (SEQ ID NO: 16) |
| Urod | ID: Hs.PT.58.26811711 | ID: Mm.PT.58.31775813 |
| | 5′-GAGTGTAGTCTGTTTCCTCTCC-3′ | 5′-GTAGTTGGACTTGACTGGACAG-3′ |
| | (SEQ ID NO: 17) | (SEQ ID NO: 19) |
| | 5′GATTGAGCTCGCAGTTACAGA-3′ | 5′-CGACCGATCTCTTCCTCAGA-3′ |
| | (SEQ ID NO: 18) | (SEQ ID NO: 20) |
| Cpox | ID: Hs.PT.58.3495748 | ID: Mm.PT.58.41976009 |
| | 5′-TGAAGTACACAGCTGATGCC-3′ | 5′-GTCCCTTCCTATGTTCCCATTG-3′ |
| | (SEQ ID NO: 21) | (SEQ ID NO: 23) |
| | 5′GCGACATGAAGACCAAGATG-3′ | 5′-GCCCCGATCATACAACAGAT-3′ |
| | (SEQ ID NO: 22) | (SEQ ID NO: 24) |
| Ppox | ID: Hs.PT.58.21362284 | ID: Mm.PT.58.42091252 |
| | 5′-CAGTCATTAACAGCAACTCCCT-3′ | 5′-TCTTCACAATCACCTAGCAAGT-3′ |
| | (SEQ ID NO: 25) | (SEQ ID NO: 27) |
| | 5′GTCCATCTACACAAGAACTGC-3′ | 5′-CTTATAATGTGGTCAGCCTCCA-3′ |
| | (SEQ ID NO: 26) | (SEQ ID NO: 28) |
| Fech | ID: Hs.PT.58.25999059 | ID: Mm.PT.58.10646197 |
| | 5′-GTGAGCAGAAAACAGAATGACC-3′ | 5′-CCATTGCTTTCACACAGTATCC-3′ |
| | (SEQ ID NO: 29) | (SEQ ID NO: 31) |
| | 5′GTGGAGCACTATTGACAGGTG-3′ | 5′-GCCCACCTCGTTATAGTATCTG-3′ |
| | (SEQ ID NO: 30) | (SEQ ID NO: 32) |
| Nrli3 | N/A | ID: Mm.PT.58.13680704 |
| | | 5′-TGCTGATTCAGTTGCAAAGATG-3′ |
| | | (SEQ ID NO: 33) |
| | | 5′-GCAGAAGTGTCTAAATGTTGGC-3′ |
| | | (SEQ ID NO: 34) |
| Nr1h4 | N/A | ID: Mm.PT.58.31148648 |
| | | 5′-TGAGCTGTGTGTTGTCTGTG-3′ |
| | | (SEQ ID NO: 35) |
| | | 5′-GGCGTTCTTGGTAATGCTTC-3′ |
| | | (SEQ ID NO: 36) |
| Abcb11 | N/A | ID: Mm.PT.58.9240423 |
| | | 5′-GAAGTCCTCTCATCTATTCGAACA-3′ |
| | | (SEQ ID NO: 37) |
| | | 5′-CACCATTCCTTTCCAAATTCCC-3′ |
| | | (SEQ ID NO: 38) |
| Abcc4 | N/A | ID: Mm.PT.58.41162071 |
| | | 5′-TGACTCACCAGTTACAGTACCT-3′ |
| | | (SEQ ID NO: 39) |
| | | 5′-TTCAGAAAACTCCGTGTAAGTCC-3′ |
| | | (SEQ ID NO: 40) |

TABLE S3
Commercial antibodies used in the study.

| | | | Catalog | Host |
|---|---|---|---|---|
| Category | Antibody | Vendor | number | Species |
| Nuclear | CAR/NR1I3 | Novus | N4111 | Mouse |
| Proteins | | Biologicals | | |
| | FXR/NR1H4 | Cell Signaling | 72105 | Mouse |
| | | Technology | | |
| | RXRα/β/γ | Santa Cruz | sc-46659 | Mouse |
| | | Biotechnology | | |
| | LAMIN B1 | Abcam | ab16048 | Rabbit |
| Heme and | ALAS-H | Santa Cruz | sc-137093 | Mouse |
| porphyrin | FECH | Biotechnology | sc-377377 | Mouse |
| biosynthesis | ALAD | Sigma-Aldrich | AV41657 | Rabbit |
| | UROD | Proteintech | 15547-1-AP | Rabbit |
| Bile acid | BSEP/ABCB11 | Abcam | ab155421 | Rabbit |
| transporters | MRP4 | Santa Cruz | sc-59614 | Rat |
| | | Biotechnology | | |
| Other proteins | p62/SQSTM1 | Abcam | ab109012 | Rabbit |
| | HRH1 | ABclonal | A1422 | Rabbit |

### REFERENCES CITED IN THE EXAMPLE 1

1. H. Puy, et al., Porphyrias. Lancet. 375, 924-937 (2010).
2. H. L. Bonkovsky, et al., Porphyrin and heme metabolism and the porphyrias. Compr. Physiol. 3, 365-401 (2013).
3. M. Balwani, et al., Clinical, biochemical, and genetic characterization of north American patients with erythropoietic protoporphyria and X-linked protoporphyria. JAMA Dermatol. 153, 789-796 (2017).
4. S. Wahlin, et al., Liver transplantation for erythropoietic protoporphyria in Europe. Liver Transpl. 17, 1021-1026 (2011).
5. C. Levy, et al., Evidence-based consensus guidelines for the diagnosis and management of protoporphyria-related liver dysfunction in erythropoietic protoporphyria and X-linked protoporphyria. Hepatology 79:731-743 (2024).
6. I. M. Heerfordt, et al., Experimental and approved treatments for skin photosensitivity in individuals with erythropoietic protoporphyria or X-linked protoporphyria: A systematic review. Biomed. Pharmacother. 158, 114132 (2023).
7. J. G. Langendonk, et al., Afamelanotide for Erythropoietic Protoporphyria. N. Engl. J. Med. 373 (1), 48-59 (2015).
8. J. S. Elenbaas, et al., A precursor-inducible zebrafish model of acute protoporphyria with hepatic protein aggregation and multiorganelle stress. FASEB J. 30, 1798-1810 (2016).
9. S. Tutois, et al., Erythropoietic protoporphyria in the house mouse. A recessive inherited ferrochelatase deficiency with anemia, photosensitivity, and liver disease. J. Clin. Invest. 88, 1730-1736 (1991).
10. S. Hanada, et al., Gender dimorphic formation of mouse Mallory-Denk bodies and the role of xenobiotic metabolism and oxidative stress. Gastroenterology. 138, 1607-1617 (2010).
11. L. Kennedy, et al., Blocking H1/H2 histamine receptors inhibits damage/fibrosis in Mdr2(−/−) mice and human cholangiocarcinoma tumorigenesis. Hepatology. 68, 1042-1056 (2018).
12. L. Pham, et al., Mast cells in liver disease progression: An update on current studies and implications. Hepatology. 75, 213-218 (2022).
13. A. H. Vo, et al., Loss of fibrinogen in zebrafish results in symptoms consistent with human hypofibrinogenemia. PLOS One. 8, e74682 (2013).
14. D. Maitra, et al., Ambient Light Promotes Selective Subcellular Proteotoxicity after Endogenous and Exogenous Porphyrinogenic Stress. J. Biol. Chem. 290, 23711-23724 (2015).
15. S. V. Weerasinghe, et al., Mouse genetic background contributes to hepatocyte susceptibility to Fas-mediated apoptosis. Mol. Biol. Cell. 27, 3005-3012 (2016).
16. J. R. Bloomer, Liver metabolism of porphyrins and haem. J. Gastroenterol. Hepatol. 13, 324-329 (1998).
17. D. Maitra, et al., Oxygen and Conformation Dependent Protein Oxidation and Aggregation by Porphyrins in Hepatocytes and Light-Exposed Cells. Cell Mol. Gastroenterol. Hepatol. 8, 659-682.el (2019).
18. N. Peitsaro, et al., Identification of zebrafish histamine H1, H2 and H3 receptors and effects of histaminergic ligands on behavior. Biochem. Pharmacol. 73 (8), 1205-1214 (2007).
19. M. Wang, et al., Integrative genomic analyses of the histamine H1 receptor and its role in cancer prediction. Int. J. Mol. Med. 33, 1019-1026 (2014).
20. C. A. Akdis, F. E. Simons, Histamine receptors are hot in immunopharmacology. Eur. J. Pharmacol. 533, 69-76 (2006).
21. E. B. Thangam, et al., The Role of Histamine and Histamine Receptors in Mast Cell-Mediated Allergy and Inflammation: The Hunt for New Therapeutic Targets. Front. Immunol. 9, 1873 (2018).
22. D. L. Marcus, et al., Effect of cimetidine on delta-aminolevulinic acid synthase and microsomal heme oxygenase in rat liver. Biochem. Pharmacol. 33, 2005-2008 (1984).
23. R. K. Leaf, A. K. Dickey, How I treat erythropoietic protoporphyria and X-linked protoporphyria. Blood. 24, 2921-2931 (2023).
24. J. Bloomer, et al., Molecular studies of liver disease in erythropoietic protoporphyria. J. Clin. Gastroenterol. 39 (4 Suppl 2), S167-S175 (2005).
25. S. Kakizaki, et al., Xenobiotic-sensing nuclear receptors CAR and PXR as drug targets in cholestatic liver disease. Curr. Drug Targets. 10, 1156-1163 (2009).
26. H. Yang, H. Wang, Signaling control of the constitutive androstane receptor (CAR). Protein Cell. 5, 113-123 (2014).
27. E. Halilbasic, et al., Bile acid transporters and regulatory nuclear receptors in the liver and beyond. J. Hepatol. 58, 155-168 (2013).
28. J. Chai, et al., Changes of organic anion transporter MRP4 and related nuclear receptors in human obstructive cholestasis. J. Gastrointest. Surg. 15, 996-1004 (2011).
29. M. Assem, et al., Interactions between hepatic Mrp4 and Sult2a as revealed by the constitutive androstane receptor and Mrp4 knockout mice. J. Biol. Chem. 279, 22250-22257 (2004).
30. J. R. Plass, et al., Farnesoid X receptor and bile salts are involved in transcriptional regulation of the gene encoding the human bile salt export pump. Hepatology. 35, 589-596 (2002).
31. M. Ratner. Patients with porphyria bask in sunlight of FDA approval. Nat. Biotechnol. 37, 1390-1391 (2019).
32. F. E. Simons, K. J. Simons, Histamine and H1-antihistamines: celebrating a century of progress. J. Allergy. Clin. Immunol. 128, 1139-1150.e4 (2011).
33. C. Koh, et al., A randomized, proof-of-concept clinical trial on repurposing chlorcyclizine for the treatment of chronic hepatitis C. Antiviral. Res. 163, 149-155 (2019).
34. J. R. Bloomer, et al., Hepatic disease in erythropoietic protoporphyria. Am. J. Med. 58, 869-882 (1975).
35. B. M. McGuire, et al., Liver transplantation for erythropoietic protoporphyria liver disease. Liver Transpl. 11, 1590-1596 (2005).
36. P. M. Farr, et al., Inhibition of photosensitivity in erythropoietic protoporphyria with terfenadine. Br. J. Dermatol. 122, 809-815 (1990).
37. S. Yamamoto, et al., Cimetidine reduces erythrocyte protoporphyrin in erythropoietic protoporphyria. Am. J. Gastroenterol. 88, 1465-1466 (1993).
38. J. H. Tu, et al., Novel Treatment Using Cimetidine for Erythropoietic Protoporphyria in Children. JAMA Dermatol. 152, 1258-1261 (2016).
39. D. Maitra, et al., Protein-aggregating ability of different protoporphyrin-IX nanostructures is dependent on their oxidation and protein-binding capacity. J. Biol. Chem. 297, 100778 (2021).
40. D. Maitra, et al., Porphyrin-Induced Protein Oxidation and Aggregation as a Mechanism of Porphyria-Associated Cell Injury. Cell Mol. Gastroenterol. Hepatol. 8, 535-548 (2019).
41. A. J. Mccullough, et al., Fecal protoporphyrin excretion in erythropoietic protoporphyria: effect of cholestyramine and bile acid feeding. Gastroenterology. 94, 177-181 (1988).
42. P. V. Tishler, B. Rosner, Treatment of erythropoietic protoporphyria with the oral sorbent colestipol: a proof-of-concept clinical trial. J. Am. Acad. Dermatol. 70, 391-392 (2014).
43. N. Fujimori, et al., Cimetidine/lactulose therapy ameliorates erythropoietic protoporphyria-related liver injury. Clin. J. Gastroenterol. 10, 452-458 (2017).
44. T. Korolnek, et al., Control of metazoan heme homeostasis by a conserved multidrug resistance protein. Cell Metab. 19, 1008-1019 (2014).
45. I. G. Chambers, et al., MRP5 and MRP9 play a concerted role in male reproduction and mitochondrial function. Proc. Natl. Acad. Sci. USA. 119, e2111617119 (2022).
46. Z. Wang, et al., Identification and characterization of a heme exporter from the MRP family in Drosophila melanogaster. BMC Biol. 20, 126 (2022).
47. D. H. Pham, et al., Deleterious Variants in ABCC12 are Detected in Idiopathic Chronic Cholestasis and Cause Intrahepatic Bile Duct Loss in Model Organisms. Gastroenterology. 161, 287-300.e16 (2021).
48. R. Kuntzman, et al., Physiological Distribution and Metabolic Inactivation of Chlorcyclizine and Cyclizine. J. Pharmacol. Exp. Ther. 149, 29-35 (1965).
49. C. Bonfils, et al., Cytochrome P-450 isozyme LM3b from rabbit liver microsomes. Induction by triacetyloleandomycin purification and characterization. J. Biol. Chem. 258, 5358-5362 (1983).
50. A. B. Nair, S. Jacob, A simple practice guide for dose conversion between animals and human. J. Basic Clin. Pharm. 7, 27-31 (2016).
51. A. Schafer, et al., Repurposing potential of 1st generation H₁-specific antihistamines as anti-filovirus therapeutics. Antiviral Res. 157, 47-56 (2018).
52. M. Yasuda, et al., Cimetidine does not inhibit 5-aminolevulinic acid synthase or heme oxygenase activity: Implications for treatment of acute intermittent porphyria and erythropoietic protoporphyria. Biomolecules 14, 27 (2023).

### Example 2. Animal Studies Using Second Generation H1 Antihistamines, or H2 Antihistamine

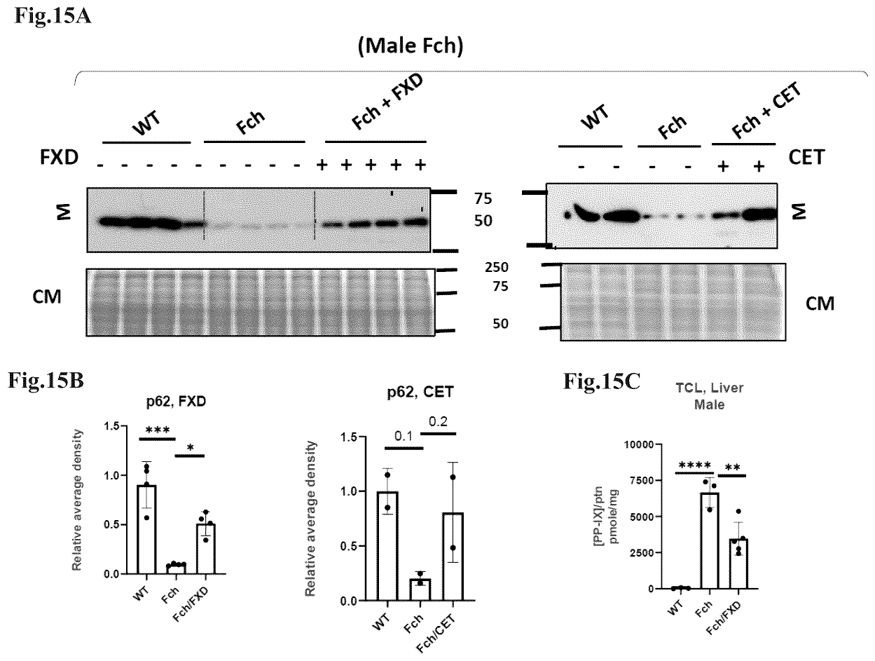

Effect of the second generation H1 antihistamines, fexofenadine (FXD) and cetirizine (CET), on protein aggregation and porphyrin accumulation in fch/fch mice was investigated. As shown in FIG. 15, overall, FXD protects the livers of fch mice from loss of p62 antibody recognition (that from Example 1 it has been shown to be due to protein oxidation). CET shows a similar trend (a larger number of experimental animals could be used to further validate the result).

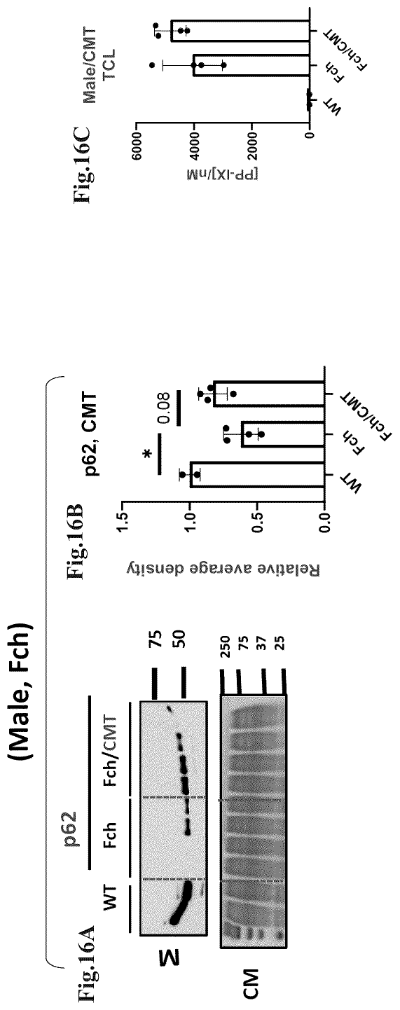

In addition, the effect of the H2 antihistamine, cimetidine (CMT), on protein aggregation and porphyrin accumulation in fch/fch mice was investigated. As shown in FIG. 16, overall, cimetidine (CMT) shows a trend in protecting the livers of fch mice from loss of p62 antibody recognition. CMT appears to function differently as compared to FXD in that CMT does not appear to decrease porphyrin accumulation in the fch mice (similar results were noted in female mice, not shown).

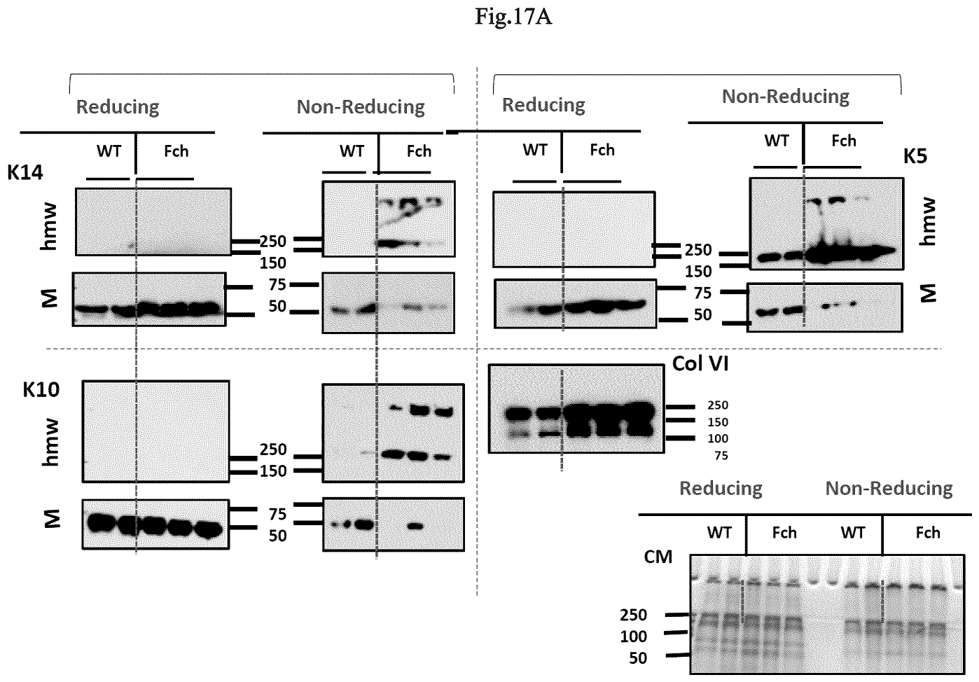

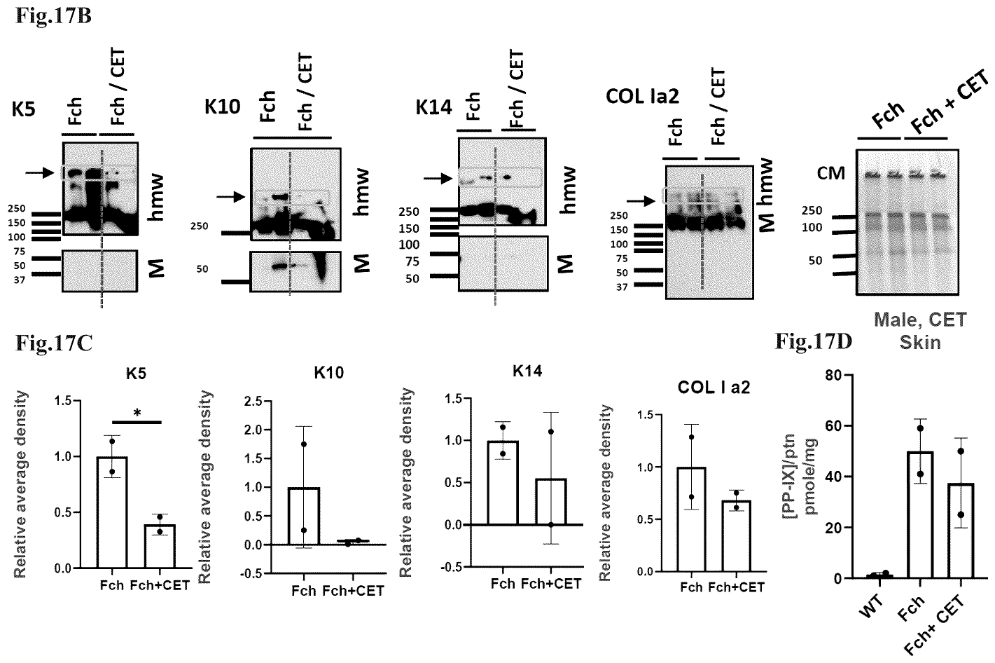

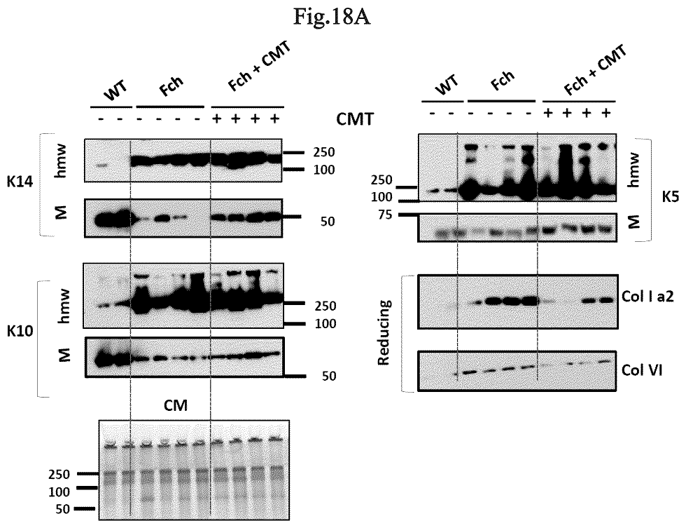

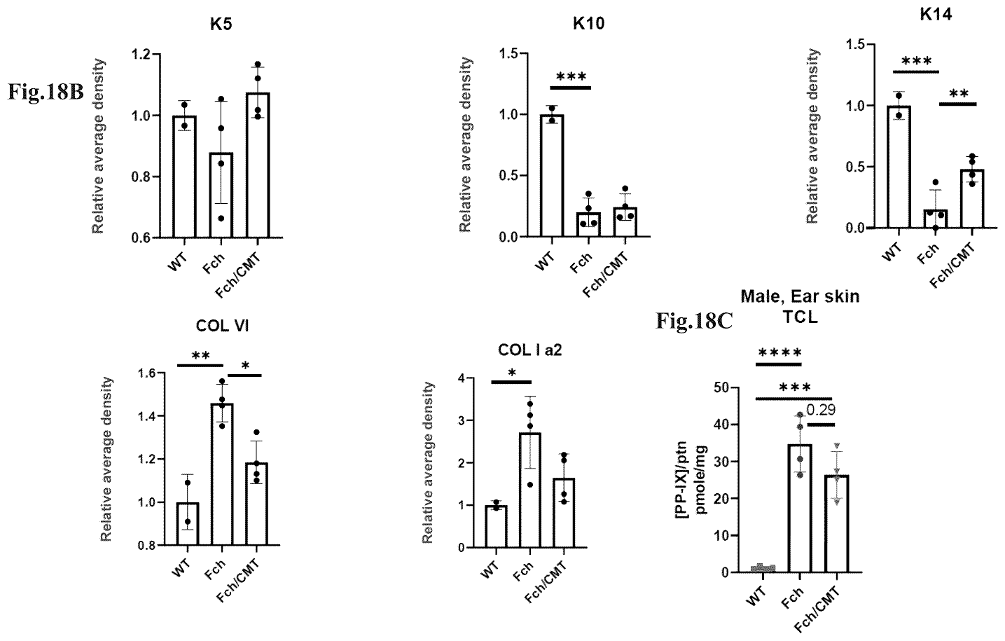

Furthermore, the effect of second generation H1 antihistamine, cetirizine (CET), on skin protein aggregation and porphyrin levels was investigated. As shown in FIG. 17, overall, Porphyrin levels are shown in the skin samples isolated from wildtype, fch, and fch mice treated with CET. Keratin K5 protein aggregation (high molecular weight) was decreased by CET. There was a trend in the decease of porphyrin level, albeit not statistically significant after treatment with CET. Analysis of additional mice could be used to validate the observed trends. The effect of the H2 antihistamine, cimetidine (CMT), on skin protein aggregation and porphyrin levels was also investigated and shown in FIG. 18.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method of treating protoporphyria related hepatobiliary disease in a subject in need of, comprising administering an H1 antihistamine to the subject.

2. The method of claim 1, wherein the H1 antihistamine is administered for at least 5 days.

3. The method of claim 1, further comprising administering a H2 antihistamine.

4. The method of claim 1, wherein protoporphyrin-IX (PP-IX) level in liver is reduced.

5. The method of claim 2, wherein PP-IX level in stool is increased.

6. The method of claim 1, wherein the H1 antihistamine is chlorcyclizine (CCZ), fexofenadine (FXF), or cetirizine (CET).

7. The method of claim 1, further comprising identifying the subject as having protoporphyria related hepatobiliary disease prior to administering the H1 antihistamine to the subject.

8. A method of treating protoporphyria in a subject in need of, comprising administering two or more antihistamines to the subject.

9. The method of claim 8, wherein the two or more antihistamines comprise a H1 antihistamine.

10. The method of claim 8, wherein the two or more antihistamines comprise an H1 antihistamine and an H2 antihistamine.

11. The method of claim 10, wherein the antihistamines are administered for at least 5 days.

12. The method of claim 10, wherein the subject has protoporphyria related hepatobiliary disease.

13. The method of claim 10, wherein the subject has protoporphyria related skin disease.

14. The method of claim 10, wherein the H1 antihistamine is chlorcyclizine (CCZ), fexofenadine (FXF), or cetirizine (CET).

15. The method of claim 10, wherein the H2 antihistamine is cimetidine (CMT) or ranitidine (RNT).

16. A method of treating protoporphyria in a subject in need of, comprising administering an H1 antihistamine to the subject, wherein the H1 antihistamine is administered for at least 5 days.

17. The method of claim 16, wherein the subject has protoporphyria related skin or liver disease.

18. The method of claim 16, wherein PP-IX level in circulating erythrocytes, serum or plasma is reduced.

19. The method of claim 16, further comprising administering an H2 antihistamine to the subject.

20. The method of claim 16, wherein the H1 antihistamine is chlorcyclizine (CCZ), fexofenadine (FXF), or cetirizine (CET).
